(19) United States
(12) Patent Application Publication
(10) Pub. No.: US 2005/0250693 A1
(43) Pub. Date: Nov. 10, 2005

(54) Metalloproteinase inhibitors

(75) Inventors: Christopher Goddard, Hawthorn (AU); Andrew Jeremy Dunbar, Coromandel Valley (AU); Matthew Paul Chen Heng Chong, Hindmarsh (AU)

Correspondence Address: LADAS & PARRY, 26 WEST 61ST STREET, NEW YORK, NY, 10023, US

(73) Assignee: GroPep Limited

(21) Appl. No.: 10/999,692

(22) Filed: Nov. 30, 2004

Related U.S. Application Data
(63) Continuation of application No. PCT/AU03/00714, filed on Jun. 6, 2003.

(30) Foreign Application Priority Data
Jun. 6, 2002 (AU) PS 2820

(51) Int. Cl. A61K 38/54

(52) U.S. Cl. 514/12

(57) ABSTRACT

The present invention provides inhibitors of metalloprotineases that may be extracted from lactational secretions such as milk and colostrum. The inhibitors have use in the treatment of a range of diseases such as disorders of the gastrointestinal tract, cardiovascular conditions and wound healing. Methods for purifiying the inhibitors are also disclosed.

### FIELD OF THE INVENTION

The present invention relates to compositions derived from a lactational secretion including milk and colostrum having anti-proteinase activity. More specifically the compositions are active against metalloproteinases and are useful in the treatment of wounds and disorders of the gastrointestinal tract.

### BACKGROUND OF THE INVENTION

No admission is made that any reference cited in this specification constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Proteinases are naturally occurring enzymes present in many tissues of the body. These enzymes act to degrade proteins, normally in a specific manner. To prevent the uncontrolled destruction of target proteins the activity of the enzymes are modulated by inhibitor substances. Thus, the combined and balanced actions of proteinases and inhibitors act to control the level of biologically active or structurally important proteins of the body, thereby regulating many important physiological processes.

One important group of proteinases are the metalloproteinases. These enzymes are characterised by their requirement for the presence of a metal ion in order to catalyse proteolysis. Approximately 17 different metalloproteinases have been identified and/or cloned which share significant sequence homology. The metalloproteinase family can be sub-divided into five groups according to their structural and functional properties: (i) the collagenases (metalloproteinases-1, 8 and 13); (ii) the gelatinases A and B (metalloproteinase-2 and metalloproteinase-9); (iii) the stromelysins 1 and 2 (metalloproteinases-3 and 10); (iv) matrilysin (MMP-7); enamelysin (MMP-20), macrophage metalloelastase (MMP-12), and MMP-19 (making up the classical metalloproteinases): and (v) the membrane-type metalloproteinases (MT-MMP-1 to 4 and stromelysin-3, MMP-11) (W. Bode et al., Ann N.Y. Acad. Sci. 878, 73, 1999). These metalloproteinases share a common multi-domain structure, but are glycosylated to different extents and at different sites. According to sequence alignment, the assembly of these domains might have been an early evolutionary event, followed by diversification. Collectively, metalloproteinases can degrade all the major components of the extracellular matrix (ECM).

The homeostasis of the ECM is controlled by a delicate balance between the synthesis of ECM proteins, production of ECM-degrading extracellular matrix metalloproteinases (MMPs), and the presence of metalloproteinase inhibitors.

One family of metalloproteinases are the tissue inhibitors of metalloproteinases (TIMPs). The TIMP family is comprised of at least four distinct members (TIMP-1 to 4) which possess 12 conserved, cysteine residues and express metalloproteinase inhibitory activity by forming non-covalent complexes with metalloproteinases. Specifically TIMPs bind to the highly conserved active zinc-binding site of the metalloproteinases in a 1:1 stoichiometry, but can also bind at other domains of metalloproteinase-2 and metalloproteinase-9. Besides their inhibitory role, TIMPs appear to have other functions that do not seem to be directly attributable to proteinase inhibition including growth factor-like, anti-angiogenic and anti-apoptotic activity.

TIMPs have been identified in a diverse range of biological tissues such as bone, amniotic fluid, cartilage, aortic endothelial cells and skin fibroblasts. These tissues require substantial purification in order to isolate metalloproteinase inhibitors, and have associated biosafety issues for human use. Furthermore these sources are only able to provide limited amounts of TIMP.

It is an aspect of the present invention to overcome or at least alleviate one or more of the difficulties or deficiencies related to the prior art by providing a plentiful source of metalloproteinase inhibitor, and methods for purifying the inhibitors. Furthermore, compositions including inhibitors are disclosed as well as methods for treating various conditions and diseases using the compositions described herein.

### SUMMARY OF THE INVENTION

In a first aspect the present invention provides a composition derived directly or indirectly from a lactational secretion of a mammal, the composition comprising an inhibitor of a metalloproteinase. Applicants have found that milk and colostrum contain useful amounts of metalloproteinase inhibitors, especially in the milk or colostrum of cows.

In a second aspect, the present invention provides a method for treating, preventing or ameliorating a disorder associated with undesirable metalloproteinase activity, the method including administering to an animal in need thereof an effective amount of a composition comprising a metalloproteinase inhibitor derived from a lactational secretion. The methods are useful in the areas of wound care, disorders of the gastrointestinal tract, and disorders of the cardiovascular system for example.

In a third aspect the present invention provides a method for at least partially purifying or enriching a metalloproteinase inhibitor, the method including the steps of
providing a lactational secretion or derivative thereof, and
subjecting the lactational secretion or derivative thereof to one or more treatment steps selected from the group consisting of centrifugation, micro-filtration, ultra-filtration, ion-exchange chromatography, molecular sieve chromatography, affinity chromatography, reverse-phase high performance liquid chromatography and transient acidification.

### DETAILED DESCRIPTION OF THE INVENTION

In a first aspect the present invention provides a composition derived directly or indirectly from a lactational secretion of a mammal, the composition comprising an inhibitor of a metalloproteinase. Applicants have shown that ungulate milk and colostrum are useful as a source of metalloproteinase inhibitors. The unexpected finding of these inhibitors in ungulate milk and colostrum provides a plentiful, renewable source of metalloproteinase inhibitor. As used herein the term “metalloproteinase” includes proteases that proteolytically degrade a component of the extracellular matrix. The term metalloproteinases includes but is not limited to (i) the collagenases (metalloproteinases-1, 8 and 13); (ii) the gelatinases A and B (metalloproteinase-2 and metalloproteinase-9); (iii) the stromelysins 1 and 2 (metalloproteinases-3 and 10); (iv) matrilysin (MMP-7); enamelysin (MMP-20), macrophage metalloelastase (MMP12), and MMP-19 (making up the classical metalloproteinases) and (v) the membrane-type metalloproteinases (MT-MMP-1 to 4 and stromelysin-3, MMP-11).

In the context of the present invention the term “lactational secretion” includes any secretion from the mammary gland of a mammal (including but not limited to milk and colostrum). The mammal may be a human, a cow, a sheep, a goat, a camel or a horse.

The present invention also includes the use of a derivative of a lactational secretion. Useful derivatives of a lactational secretion include any product in which the proportions of fat and/or protein constituents thereof are altered, including but not limited to cheese whey, skim milk, acid (casein) whey, dried milk powder, colostral whey and defatted colostrum. Derivatives also include process intermediates such as chromatography eluates, filtrates, retentates and the like.

Preferably the mammal is an ungulate animal. Preferably the ungulate animal is a cow. Compared with other ungulates, cows produce large volumes of milk on a regular basis ensuring a relatively steady and renewable supply of metalloproteinase inhibitor. Cows that have recently calved produce large volumes of colostrum.

Preferably the inhibitor of metalloproteinase is present at a concentration ranging from about 0.01 μg/ml to about 100 mg/ml in the composition. More preferably the inhibitor of metalloproteinase is present at a concentration ranging from about 0.1 μg/ml to about 1000 μg/ml. Even more preferably the inhibitor of metalloproteinase is present at a concentration ranging from about 1 μg/ml to 500 μg/ml. In a highly preferred embodiment, the inhibitor of metalloproteinase is present at a concentration of about 11 μg/ml, or about 45 μg/ml or about 50 μg/ml as quantified by a fluorescence-quenching substrate assay as described in Example 3.

In a preferred form of the invention the inhibitor is a tissue inhibitor of a metalloproteinase (TIMP). As used herein the term “tissue inhibitor of a metalloproteinase” includes but is not limited to polypeptides which regulate the activity of metalloproteinases which includes TIMP-1, TIMP-2, TIMP-3 and TIMP-4. The TIMP family is comprised of at least four distinct members (TIMP-1 to 4) which possess 12 conserved cysteine residues and express metalloproteinase inhibitory activity by forming non-covalent complexes with metalloproteinases. Specifically TIMPs bind to the highly conserved active zinc-binding site of the metalloproteinases in a 1:1 stoichiometry, but can also bind at other domains of metalloproteinase-2 and metalloproteinase-9. Besides their inhibitory role, TIMPs appear to have other functions that do not seem to be directly attributable to protease inhibition including growth factor-like, anti-angiogenic and anti-apoptotic activity. TIMPs are expressed by a variety of cell types and are present in most tissues. Whilst TIMPs have been described in some body fluids there are no reports describing the presence of metalloprotease inhibitors in ungulate lactational secretions.

TIMP-1 was originally isolated from rabbit bone and characterised as a collagenase inhibitor (A. Sellers and J. J. Reynolds. Biochem. J. 167, 353, 1977). Subsequently, human TIMP-1 was purified from amniotic fluid (G. Murphy et al., Biochem. J. 195, 167, 1981) and skin fibroblasts and has since been purified from a number of other sources (for a review see J. Woessner Methods in Mol. Biol. 151, 1, 2001). Human TIMP-1 consists of 184 amino acids which include 12 cysteine residues forming six disulphide bonds and a characteristic six-loop structure. Human TIMP-1 is extensively glycosylated with a molecular mass of approximately 28.5 kDa, although it can range from 30 to 34 kDa, depending on the degree of glycosylation.

Human TIMP-2 is a 21 kDa unglycosylated protein which was initially identified as a protein that co-purified with the 72 kDa progelastinase (MMP-2) in supernatants of human melanoma cells and fibroblasts (G. I. Goldberg et al., Proc. Natl. Acad. Sci. (USA) 86, 8207, 1989; W. Stetler-Stevenson et al., J. Biol. Chem. 264, 374, 1989) and in the conditioned medium of alveolar macrophages (S. D. Shapiro et al., J. Biol. Chem. 267, 1992). Human TIMP-2 is 40% identical to human TIMP-1 at the amino acid level and contains the conserved six disulphide bond structure.

Bovine TIMP-2, was originally isolated from bovine cartilage and characterised as a collagenase inhibitor (J. Murray et al., J. Biol. Chem. 261, 4154, 1986). Subsequently, TIMP-2 was isolated from the conditioned medium of bovine aortic endothelial cells (Y. De Clerck et al., J. Biol. Chem. 264, 17445, 1989) which led to the cloning and characterisation of the bovine TIMP-2 cDNA sequence from these cells (T. Boone et al., Proc. Natl. Acad. Sci. (USA) 87, 2800, 1990). The bovine TIMP-2 cDNA encodes a leader sequence of 26 amino acids and mature protein sequence of 194 amino acids. Bovine TIMP-2 is 94% identical to human TIMP-2 at the amino acid level and shares 41% and 42% identity with bovine TIMP-1 and TIMP-3, respectively. Refer to Table 1 for the amino acid sequences for bovine TIMP-2 and human TIMP 1, 2, 3 and 4.

TABLE 1
Amino acid sequences for TIMPs.

Bovine TIMP-2
NH₂₋
CSCSPVHPQQAFCNADIVIRAKAVNKKEVDSGNDIYGNPIKRIQYEIKQI
KMFKGPDQDIEFIYTAPAAAVCGVSLDIGGKKEYLIAGKAEGNGNMHITL
CDFIVPWDTLSATQKKSLNHRYQMGCECKITRCPMIPCYISSPDECLWMD
WVTEKNINGHQAKFFACIKRSDGSCAWYRGAAPPKQEFLDIEDP-COOH
Human TIMP-1
NH₂₋
CTCVPPHPQTAFCNSDLVIRAKFVGTPEVNQTTLYQRYEIKMYKMYKGFQ
ALGDAADIRFVYTPAMESVCGYFHRSHNRSEEFLIAGKLQDGLLHITTCF
SVAPWNSLSLAQRRGFTKTYTVGCEECTVFPCLSIPCKLQGTHCLWTDQL
LQGSEKGFQSRHLACLPREPGLCTWQSLRSQIA-COOH
Human TIMP-2
NH₂₋
CSCSPVHPQQAFCNADVVIRAKAVSEKEVDSGNDIYGNPIKRIQYEIKQI
KMFKGPEKDIEFIYTAPSSAVCGVSLDVGGKKEYLIAGKAEGDGKMHITL
CDFIVPWDTLSTTQKKSLNHRYQMGCECKITRCPMIPCYISSPDECLWMD
WVTEKNINGHQAKFFACIKRSDGSCAWYRGAAPPKQEFLDIEDP-COOH
Human TIMP-3
NH₂₋
CTCSPSHPQDAFCNSDIVIRAKVVGKKLVKEGPFGTLVYTIKQMKMYRGF
TKMPHVQYIHTEASESLCGLKLEVNKYQYLLTGRVYDGKMYTGLCNFVER
WDQLTLSQRKGLNYRYHLGCNCKIKSCYYLPCFVTSKNECLWTDMLSNFG
YPGYQSKHYACIRQKGGYCSWYRGWAPPDKSIINATDP-COOH
Human TIMP-4
NH₂₋
CSCAPAHPQQHICHSALVIRAKISSEKVVPASADPADTEKMLRYEIKQIK
MFKGFEKVKDVQYIYTPFDSSLCGVKLEANSQKQYLLTGQVLSDGKVFIH
LCNYIEPWEDLSLVQRESLNHHYHLNCGCQITTCYTVPCTISAPNECLWT
DWLLERKLYGYQAQHYVCMKHVDGTCSWYRGHLPLRKEFVDIVQP-COOH

TIMP-3 was initially identified and purified from chicken embryo fibroblasts as a 21 kDa unglycosylated protein (N. Pavloff et al., J. Biol. Chem. 267, 17321, 1992). The human homolog was subsequently detected as a serum-inducible protein in WI-38 fibroblasts (M. Wick et al., J. Biol. Chem. 269, 18953, 1994). Human TIMP-3 is 30% and 38% homologous to human TIMP-1 and human TIMP-2, respectively. More recently, a fourth member of the TIMP family, TIMP-4 has been identified by molecular cloning (J. Greene et al., J. Biol. Chem. 271, 30375, 1996). Human TIMP-4 has a predicted molecular weight of 22 kDa and the recombinant form of the protein has a molecular mass of 24 kDa.

In a more preferred form of the invention the inhibitor is a bovine TIMP-2 polypeptide or a functional equivalent thereof. As used herein the term “functional equivalent” includes molecules having a substantially similar ability as TIMP-2 to inhibit the activity of metalloproteases. Functional equivalents include allelic variants of TIMP-2 resulting from at least one mutation in the nucleic acid sequence and which may result in altered mRNA and may or may not result in polypeptides altered in structure or function. Alleles of a gene may arise as a result of natural deletions, substitutions, rearrangements or additions of nucleotides.

In the context of “allelic variant”, the term “variant” refers to an amino acid sequence that is altered by one or more amino acids. The variant may have conservative changes wherein a substituted amino acid has similar structural or chemical properties, for example replacement of isoleucine with leucine. A variant may have nonconservative changes wherein a substituted amino acid has different structural or chemical properties, for example replacement of alanine with glycine. The term “variant” also includes modifications in glycosylation, either as well as other variations including allelic variations. Thus, the modified proteins including these amino acid sequences will usually be substantially equivalent to these proteins in either function or structure and as such are defined as analogues. Preferably, the metalloproteinase inhibitor variant has the 12 cystine residues of the native amino acid sequence conserved to enable the polypeptide to form non-covalent complexes with metalloproteinases. Even more preferably, the metalloproteinase inhibitor variant is TIMP-2 which has the 12 cystine residues of the native amino acid sequence conserved to enable the polypeptide to form non-covalent complexes with metalloproteinases.

Preferably the TIMP-2 has a molecular weight of 21,000 Da as determined by SDS-PAGE. In a further preferred form the TIMP-2 has an isolectric point of about 7.0.

Preferably the TIMP-2 includes the N terminal sequence NH2-CSCSPVHP. In a highly preferred form the TIMP-2 has the following sequence
NH2_CSCSPVHPQQAFCNADIVIRAKAVNKKEVDSGNDIYGNPIKRIQYEIKQIK MFKGPDQDIEFIYTAPAAAVCGVSLDIGGKKEYLIAGKAEGNGNMHITLCDFIVP WDTLSATQKKSLNHRYQMGCECKITRCPMIPCYISSPDECLWMDWVTEKNIN GHQAKFFACIKRSDGSCAWYRGAAPPKQEFLDIEDP_COOH

Preferably the inhibitor is capable of inhibiting metalloproteinase 2 and/or metalloproteinase 9. Metalloproteinase 2 is also known as gelatinase A. Metalloproteinase 2 is a proteolytic enzyme having a molecular weight of 72 kDa which catalyses the degradation of collagen type IV by acting on the peptide bonds. Metalloproteinase 9 is also known as gelatinase B. Metalloproteinase 2 is a proteolytic enzyme having a molecular weight of 92 kDa which catalyses the degradation of collagen type IV by acting on the peptide bonds.

Preferably the inhibitor is capable of inhibiting membrane type matrix metalloproteinases but is not capable of inhibiting tumour necrosis factor-alpha converting enzyme.

In view of the low sequence homology between the TIMPs, it is not surprising that the four members identified to date have distinct biological functions. Importantly, TIMP-2 and TIMP-3, unlike TIMP-1 are effective inhibitors of the membrane-type MMPs (MT-MMPs), while TIMP-3 but not TIMP-1, TIMP-2 or TIMP-4 inhibits the activity of tumor necrosis factor-alpha converting enzyme (TACE) (Amour et al., FEBS Lett. 435 39-44, 1998).

Preferably the inhibitor is not a growth factor and/or is not capable of stimulating the proliferation of rat L6 myoblasts.

In a further preferred form the composition includes one or more cell growth stimulating factors having an approximately neutral to basic isoelectric point and/or is capable of stimulating rat L6 myoblasts.

Preferably the growth factor is selected from the group including transforming growth factor beta, insulin-like growth factor I, insulin-like growth factor II, betacellulin, any member of the fibroblast growth factors as described in the art form example fibroblast growth factor I or fibroblast growth factor II, insulin-like growth factor binding proteins 1, 2, 3, 4, 5 or 6 and platelet-derived growth factor.

In a preferred form of the invention the lactational secretion is first processed to cheese whey before use in the composition.

Cheese whey is a by-product of the cheese industry that has had essentially all the fat and casein removed during cheese manufacture. At the present state of the art cheese whey is essentially valueless, and indeed it may represent a net cost to the industry since it is a potential pollutant. It is a low protein, high salt product available in large amounts. The main protein constituents present in cheese whey are alpha lactalbumin (αLA) and beta lactoglobulin (βLG), which usually account for more than 90% of the proteins present. Significant amounts of serum albumin, immunoglobulins and residual casein may also be present.

The composition may include one or more carriers and/or excipients. In a preferred form of the invention the carrier and/or excipient are veterinarily, nutriceutically, pharmaceutically or cosmetically acceptable. Pharmaceutical and cosmetic compositions according to the present invention may be adapted for administration in any suitable manner. The composition may be adapted for internal or topical administration. The composition may be in an oral, injectable, topical or suppository form. Preferred delivery routes include, dermal, intravaginal, intravenous, respiratory, and gastrointestinal delivery. It is to be understood that the compositions as described herein are not limited to use with humans, and include any animal that could benefit from the compositions.

Methods and pharmaceutical carriers for preparation of pharmaceutical compositions, including compositions for topical administration are well known in the art, as set out in textbooks such as Remington's Pharmaceutical Sciences, 18th Edition, Mack Publishing Company, Easton, Pa., USA.

Compositions of the present invention may be formulated so that they are suitable for oral administration. The compositions may be presented as discrete units such as capsules, sachets or tablets each containing a predetermined amount of the active ingredient; as a powder or granules; as a solution or a suspension in an aqueous or non-aqueous liquid; as a mouthwash or as an oil-in-water liquid emulsion or a water-in-oil liquid emulsion. The active ingredient may also be presented as a bolus, electuary or paste.

It should be understood that in addition to the ingredients particularly mentioned above, the compositions of this invention may include other agents conventional in the art having regard to the type of composition in question, for example, those suitable for oral administration may include such further agents as sweeteners, thickeners and flavoring agents.

In a preferred form of the invention the compositions of the present invention include a carrier selected from the group consisting of a synthetic or biological polymer, glycosaminoglycan, or extracellular matrix molecule including fibrin, collagen, gelatin, a synthetic polymer, agarose, an alginate, methylcellulose, hyaluronic acid, a hydrocolloid, an alginate, saline solution, powder, ointment, salve or irrigant or incorporated or impregnated into a dressing (absorbable and non-absorbable), a transdermal patch or releasable dressing associated with gauze, a bandage, suture, plaster, staple, prosthetic device, screw or plate (biodegradable or non-biodegradable), toothpaste, gum or resin for chewing, mouth wash or gel. The skilled artisan will be familiar with the appropriate carrier to use depending on the route or means for administration.

In another preferred form the composition has at least one further active ingredient selected from the group including antibiotics, anti-inflammatories, antiseptics, other growth promotants, anaesthetics. The compositions described herein may have other molecules associated therewith to aid releasability, stability, solubility, activity and/or association with the wound support, including adjuvants, carriers, solubilizing agents, and growth factors as discussed above. Furthermore the lactational secretion or derivative thereof or compositions of the present invention may be used in combination with other compounds or molecules which act in synergistic, agonistic and/or additive concert. There are no limitations to the nature of these ingredients except they should be pharmacologically and physiologically acceptable for administration and should not degrade the activity, or render harmfully toxic the active ingredients.

The compositions of the present invention may be useful in combination with known therapeutic agents. If formulated as a fixed dose, such combination products may employ the inhibitor in an appropriate dosage range and the other pharmaceutically active agent within its approved dosage range. The compositions may be used sequentially with known therapeutic agents when a combination formulation is inappropriate.

The compositions of the present invention may also be useful in nutriceutical or cosmetic applications and as such may be combined with other ingredients as are commonly used in products for those applications. The term nutriceutical is generally intended to mean any nutritional supplement designed for any specific clinical purpose, as foods for general consumption or to be used as supplements to diet.

In a preferred form of the invention the anti-inflammatory is capable of at least partially inhibiting the activity of tumour necrosis factor alpha. In a more preferred form, the anti-inflammatory is an antibody capable of binding to soluble tumour necrosis factor alpha such as REMICADE™ or a soluble receptor such as ENBREL™.

In a preferred embodiment, the composition comprises a plurality of basic cell growth stimulating factors with a property of stimulating the proliferation of rat L6 myoblasts. Even more preferably, the composition comprises growth factors selected from the group including; transforming growth factor beta, insulin-like growth factor I, insulin-like growth factor II, betacellulin, any member of the fibroblast growth factors as described in the art for example fibroblast growth factor I or fibroblast growth factor 11, insulin-like growth factor binding proteins 1, 2, 3, 4, 5 or 6 and platelet-derived growth factor.

In a further preferred embodiment the composition is enriched for a metalloproteinase inhibitor. As used herein the term “enriched” means having a higher proportion of a given component as compared with the lactational secretion from which the composition is derived.

Preferably the TIMP-2 in the milk or colostrum or milk product is present in the percent purity ranging from 0.01 to 10%. In a further preferred form the TIMP-2 in the milk or colostrum or milk product is present in the percent purity ranging from 10 to 30%. In yet a further preferred form the TIMP-2 in the milk or colostrum or milk product is present in the percent purity ranging from 30 to 70%. In yet a further preferred form the TIMP-2 is present in the percent purity ranging from 70 to 99.9%.

Preferably the composition has a neutral to basic pH.

In a preferred aspect of the invention the composition has one or more of the following properties: reduced amounts of alpha lactalbumin, beta lactoglobulin and casein compared with the milk product; less than about 1% w/w of salt present in milk product, less than 5% of the casein, alpha lactalbumin, beta lactoglobulin, immunoglobulin and/or albumin in the milk product.

In a second aspect, the present invention provides a method for treating, preventing or ameliorating a disorder associated with undesirable metalloproteinase activity, the method including administering to an animal in need thereof an effective amount of a composition comprising a metalloproteinase inhibitor derived from a lactational secretion as described herein.

Preferably the method is a method for treating a wound. There are no limitations to the type of surface wound that may be treated, and these include, but are not limited to ulcers, conditions that result from surgery, therapeutically induced wounds, wounds associated with disorders of the central nervous system, any exfoliative disease of the skin, wounds associated with local or systemic infection, congenital wounds, pathological wounds, traumatic and accidental wounds, and burns. In a preferred form, the surface would exhibits undesirable metalloproteinase activity. In a preferred form the skin of the wound is not intact.

In the above methods for treating wounds, lactational secretions or derivative thereof or compositions of the present invention may be applied directly to wounds in a biologically acceptable carrier to ensure sustained release at sufficient concentration in the wound environment. In treating a wound, the metalloproteinase inhibitors may be associated with a wound support. As used herein the term “wound support” includes any means which is used to support or secure a wound and includes a surgical securing means. The term includes plasters, dressings, sutures, staples and the like. The wound to be supported may be a wound created by surgery, or the result of accident or other injury. The lactational secretion or derivative thereof or composition may be present on the surface of the wound support or may be impregnated in the wound support and is able to be released therefrom.

Preferably the wound is an ulcer caused by pressure, vascular disease, diabetes, autoimmune disease, sickle cell diseases or hemophilia; a result of surgery; therapeutically induced; associated with disorders of the central nervous system, and resulting from any exfoliative disease of the skin; a associated with either local or systemic infection such as yaws, HIV, chicken pox or herpes infection; congenital; a corneal injury to the eye; a pathological wound; a traumatic or accidental wound; or a burn.

In a preferred method the concentration of the metalloproteinase inhibitor is from about 0.1 ng/ml to about 10 μg/ml of fluid in the local environment at the wound site. Even more preferably the concentration of the metalloproteinase inhibitor is from about 1 ng/ml to about 1 μg/ml of fluid in the local environment at the wound site.

The present invention also provides a method for preventing, ameliorating or treating a condition associated with a gastrointestinal injury, disease or ulcer, the method including administering to an animal in need thereof an effective amount of composition as described herein. In a preferred method the concentration of the metalloproteinase inhibitor is from about 0.1 μg/ml to about 1000 μg/ml, even more preferably about 1 g/ml to about 500 μg/ml.

As used herein the term “gastrointestinal injuries, diseases or ulcers” includes the following types of damage to or diseases of the gastrointestinal tract:
(a) dental and oral wounds, including those associated with periodontal disease;
(b) peptic ulceration of the duodenum, stomach or esophagus including gastric ulcers caused by radiation, non-steroidal anti-inflammatory drug (NSAID) therapy, helicobacter pylori bacteria or chemotherapy
(c) inflammatory bowel diseases such as ulcerative colitis or Crohn's disease;
(d) ulcers associated with stress conditions, for example burns, trauma, sepsis, shock, intracranial surgery or head surgery;
(e) damage to the lining of the alimentary tract, including mucositis, resulting from radiotherapy and/or chemotherapy with agents such as mechlorethamine, melphalan, busulphan, cytarabine, floxuridine, 5-fluorouracil, mercaptopurine, methotrexate, thioguanine, bleomycin, actinomycin-D, daunorubicin, etoposide, mitomycin, vinblastine, vincristine, hydroxyurea or procarbazine;
(f) inadequate gut function or damage to the gut associated with prematurity such as necrotizing enterocolitis or poor gut motility;
(g) diarrhoeal conditions such as associated with bacterial, viral, fungal or protozoan infection, including AIDS;
(h) food intolerances such as coeliac disease;
(i) cancers of the gastrointestinal tract, including buccal cavity, esophagus, stomach or bowel;
(j) surgically induced damage such as following partial gut resection, short gut syndrome, jejunostomy, ileostomy, colostomy;
(k) damage due to esophageal reflux;
(l) conditions associated with loss of gut barrier function such as external burns, trauma, sepsis or shock;
(m) congenital conditions resulting in inadequate gastrointestinal function or damage such as volvulus and cystic fibrosis; and
(n) autoimmune diseases that affect the gut, such as Sjogren's Syndrome.

In the context of the invention, the term “effective amount” as used herein means an amount sufficient to elicit a statistically significant response at a 95% confidence level (p<0.05 that the effect is due to chance alone). Preferably, an effective amount is that amount to at least partially attain the desired response of a reduction in metalloproteinase activity.

The compositions may be administered in therapeutically effect amounts. A therapeutically effective amount means the amount required at least partly to attain the desired effect, ie to alleviate or prevent the symptoms of undesirable metalloproteinase activity, or alternatively to delay the onset of, inhibit the progression of, or halt altogether, the onset or progression of the undesirable metalloproteinase activity, or to reduce metalloproteinase activity. Preferably the term “therapeutically effective amount” as used herein means amount sufficient to elicit a statistically significant response at a 95% confidence level (p<0.05 that the effect is due to chance alone).

Such amounts will depend, of course, on the particular condition being treated, the severity of the condition, and individual patient parameters, including age, physical condition, size, weight and other concurrent treatment, and will be at the discretion of the attending physician. These factors are well known to those of ordinary skill in the art, and can be addressed with no more than routine experimentation. It is generally preferred that a minimum effective dose be determined according to sound medical judgment. It will be understood by those of ordinary skill in the art, however, that a higher dose may be administered for medical, psychological or other reasons.

Symptomatic patients may be identified after a careful history of the above symptoms in the injury, disease of ulcer and testing for metalloproteinase activity with a group of investigations.

There are no limitations to the type of gastrointestinal injuries, diseases or ulcers that may be treated, and these include, but are not limited to dental and oral wounds, peptic ulcers, inflammatory bowel diseases, ulcers associated with stress conditions, damage caused by radiotherapy and/or chemotherapy, inadequate gut function or damage associated with prematurity, diarrhoeal conditions, damage caused by food intolerance, cancer of the gastrointestinal tract, surgically induced damage, damage caused by esophageal reflux, conditions associated with loss of gut barrier function, congenital conditions resulting in inadequate gastrointestinal function or damage, and autoimmune diseases that affect the gut.

The composition may be administered at any appropriate time including prior to, during or after the gastrointestinal injury, disease or ulcer has become evident.

In a preferred method the condition is a dental or oral wound; peptic ulceration of the duodenum, stomach or esophagus; inflammatory bowel disease; an ulcer associated with stress conditions; damage to the lining of the alimentary tract; inadequate gut function or damage to the gut associated with prematurity; a diarrheal condition; a food intolerance; a cancer of the gastrointestinal tract; surgically induced damage to the gut; damage due to esophageal reflux; a condition associated with loss of gut barrier function; a congenital condition resulting in inadequate gastrointestinal function or damage; or an autoimmune disease that affects the gut.

In a fifth aspect the present invention provides a method for preventing, ameliorating and/or treating disorders associated with undesirable metalloproteinase activity, the method including administering to an animal in need thereof an effective amount of a lactational secretion or derivative thereof or composition described herein. As used herein the term “disorders associated with metalloproteinase activity” includes the following:
(i) disorders of the cardiovascular system, where undesirable metalloproteinase activity has effected the remodeling of the cardiovascular system, including dilated cardiomyopathy, congestive heart failure, atherosclerosis, plaque rupture, reperfusion injury, ischemia, chronic obstructive pulmonary disease, angioplastly restenosis and aortic aneursm;
(ii) disorders of others tissues, where metalloproteinases are involved in the irregular remodeling including disorders of bone such as osteosclerosis or osteoporosis, disorders of other tissues such as liver cirrhosis and fibrotic lung disease, disorders of nervous tissues such as multiple sclerosis;
(iii) disorders relating to viral infection whereby metalloproteinase activity is altered, such as cytomegalovirus, retinitis, HIV and the resulting syndrome AIDS.
(iv) disorders relating to inflammation involving the implication of metalloproteinases such as inflammatory bowel disease, Crohn's disease, ulcerative colitis, pancreatitis, diverticultitis, asthma or related lung disease, rheumatoid arthritis, gout, Reiter's Syndrome, lupus erthmatosis, ankylosing spondylitis, autoimmune keratitis, pulmonary disease, bronchitis, emphysema, cystic fibrosis, acute respiratory distress syndrome;
(v) disorders relating to skin involving the implication of metalloproteinases, including psoriasis, scleroderma and atopic dermatitis or disorders relating to ultraviolet damage of skin which results in the skin having an aged and/or wrinkled appearance.

Symptomatic patients are identified after a careful history of the above symptoms in tissue affected and testing for metalloproteinase activity with a group of investigations.

In a preferred method the condition is a disorder of the cardiovascular system including but not limited to dilated cardiomyopathy, congestive heart failure, atherosclerosis, plaque rupture, reperfusion injury, ischemia, chronic obstructive pulmonary disease, angioplastly restenosis, aortic aneurism; a disorder of a tissue where a metalloproteinase is involved in the irregular remodeling including disorders of bone, liver, lung and nervous tissues; a disorder relating to viral infection whereby metalloproteinase activity is altered; a disorder relating to inflammation involving the implication of metalloproteinases; a disorder relating to skin involving the implication of a metalloproteinase, including but not limited to psoriasis, scleroderma and atopic dermatitis or disorders relating to ultraviolet damage of skin which results in the skin having an aged and/or wrinkled appearance.

For all methods of treatment described herein the daily dosage can be routinely determined by the attending physician or veterinarian. Generally the dosage will vary according to the age, weight, and response of the individual patient, as well as the severity of the patient's symptoms. In general a suitable dose of the inhibitor of the invention will be in the range of about 0.1 μg to about 100 mg per kilogram body weight of the recipient per day, preferably in the range of about 1 μg to about 50 mg per kilogram body weight per day. However, the dose will also depend on the formulation and purity of the lactational secretion or derivative thereof that is used.

In a third aspect. The present invention provides a method for at least partially purifying or enriching a metalloproteinase inhibitor, the method including the steps of
providing a lactational secretion or derivative thereof, and
subjecting the lactational secretion or derivative thereof to one or more treatment steps selected from the group consisting of centrifugation, micro-filtration, ultra-filtration, ion-exchange chromatography, molecular sieve chromatography, affinity chromatography, reverse-phase high performance liquid chromatography and transient acidification.

The starting material may be a milk product filtrate substantially free of insoluble material. Skim milk typically has higher protein and fat concentrations and lower salt concentrations than cheese whey. Skim milk also contains higher amounts of insoluble protein, especially particulate casein. In order to obtain a suitable composition from skim milk, a filtration step before contacting the skim milk with the cation-exchange resin is typically required unless the selected cation-exchange resin has flow and adsorption characteristics that make it suitable for use with fat-containing and particulate-containing fluids.

The lactational secretion or derivative thereof may be clarified by centrifugation or filtration, such as by filtration through a suitable sieve. The lactational secretion or derivative thereof may be filtered through a hollow fiber cartridge of defined porosity.

Colostrum is the fluid produced during the first few days of lactation. Colostrum has a much higher protein concentration than skim milk or cheese whey. The higher protein concentration of colostrum could facilitate the isolation of metalloproteinase inhibitors because much smaller volumes need to be applied to the cation-exchange resin. However, colostrum has a high fat content and in many mammalian species contains high concentrations of immunoglobulins. These two aspects make it technically more difficult to isolate metalloproteinase inhibitors on a large scale because, firstly, the preliminary filtration step in the process will need to be particularly efficient, and secondly, proportionally more cation-exchange resin can be required because immunoglobulins can be adsorbed to the resin.

A number of treatment steps may be used to purify, enrich or activate metalloproteinase inhibitors. The suitability of a treatment step can be evaluated by subjecting a lactational secretion or derivative thereof to a purification step which is advantageous for the adsorption or separation, and measuring the proportion of metalloproteinase inhibitors,

In a preferred embodiment the treatment steps include cation exchange chromatography followed by ultrafiltration. The suitability of the cationic exchange resin can be evaluated by passing a milk product through a column of the resin to be tested at neutral pH, or at another defined pH that is advantageous for the adsorption or separation, and measuring the proportion of metalloproteinase inhibitors, and in particular TIMP-2, using a gelatinase activity assay, migration as a single band of approximately 21,000 Da following SDS-PAGE, N-terminal sequence analysis or mass spectrometry or like assay available in the prior art. Preferably, the cation-exchange resin has a suitable pore size and a suitable functional group to selectively adsorb the metalloproteinase inhibitors.

A Sepharose-based cation exchange gel may be used. Sepharose is a trade name for a family of agarose-based cationic exchange resins.

The desorption of the metalloproteinase inhibitors from the ion exchange resin leads to a preparation enriched in metalloproteinase inhibiting properties. The eluate may be concentrated and filtered utilizing any suitable technique. The eluate may be concentrated for example by conventional ultrafiltration or further chromatography methods or other procedures to yield a composition including an inhibitor of a metalloproteinase.

The method can include for example, additional steps such as treating the lactational secretion or derivative thereof sequentially by subjecting the milk product to a clarification step to remove insoluble materials therefrom; adjusting the pH of the clarified product to between approximately 6.5 to 8.0; contacting the clarified milk product with a suitable cationic-exchange resin; eluting from the cation exchange resin at high ionic strength or high pH with a suitable buffer solution to provide an eluate; and subjecting the eluate to a concentration step and diafiltration step to remove salt therefrom.

The elution from the cationic-exchange resin is achieved at high ionic strength such that the metalloproteinase inhibitors are recovered. For example, the metalloproteinase inhibitors adsorbed to the agarose-based resin can be eluted with 1M NaCl containing 0.25M NH₄OH.

The concentration step can include ultrafiltration. For example a 3000 Da excluding membrane can be used. The diafiltration step used to remove salt from the eluate can include a diafiltration against 150 mM NaCl or a volatile salt, for example, ammonium bicarbonate.

For example, the method for preparing a composition including an inhibitor of a metalloproteinase may include treating a lactational secretion or derivative thereof sequentially by subjecting the secretion or derivative to a filtration step to remove insoluble materials therefrom; adjusting the pH of the filtrate to between approximately 6.5 to 8.0; contacting the filtrate with a suitable cationic exchange resin so that metalloproteinase inhibitors with basic to neutral isoelectric points are adsorbed thereto; eluting the cationic-exchange resin with the buffer solution to provide an eluate; and treating the eluate to remove salt therefrom.

Preferably TIMP-2 with an isoelectric point of 7.0, is adsorbed in sufficient quantities such that the activity of the metalloproteinase inhibitors are detected using the metalloproteinase inhibitory activity of metalloproteinase 2 and metalloproteinase 9 as detected using a gelatinase activity assay and wherein the major proteins with acidic isoelectric points in the lactational secretion or derivative thereof are not absorbed; eluting from the cationic exchange with a suitable buffer solution; subjecting the eluate to a concentration step and diafiltration step to remove salt therefrom to obtain a composition including an inhibitor of a metalloproteinase.

Preferably, the ultrafiltration step results in the concentration of a metalloproteinase inhibitor.

Preferably a permeate or retentate containing a metalloproteinase inhibitor resulting from the ultrafiltration step is subjected to transient acidification.

The method can also further include for example, an acidification step. Preferably the acidification is conducted at a pH below approximately below pH 3.0. More preferably, the composition including an inhibitor of a metalloproteinase is acidified to a pH in the range of 2.0 to 3.0. An acidification pH of about 2.5 is particularly preferred.

Preferably acidification is carried out using an inorganic acid, for example, HCl. Acidification may be achieved by dissolving the composition including an inhibitor of a metalloproteinase, in water and acidifying with a strong inorganic acid such as 5M HCl and drying.

The process of the invention may include the further step of removing inactive proteins after acidification has taken place. Removal of inactive proteins may be carried out using molecular sieve chromatography or ultrafiltration under acidic conditions. For example a molecular sieve chromatography process may be used to obtain a composition including metalloproteinase inhibitors having molecular weights of 21,000 Da.

For example, the method for preparing a composition including an inhibitor of a metalloproteinase may include treating milk produce sequentially by subjecting the milk product to a filtration step, to remove insoluble materials therefrom; adjusting the pH of the filtrate to between approximately 6.5 to 8.0; contacting the filtrate with a suitable cationic exchange resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay and wherein the major proteins with acidic isoelectric points in the milk product are not absorbed; eluting from the cationic exchange with a suitable buffer solution; subjecting the eluate to a concentration step and diafiltration step to remove salt therefrom; providing a source of acid; subjecting the composition including an inhibitor of a metalloproteinase to transient acidification.

In a preferred form of the invention the acidified permeate or retentate is subjected to gel filtration chromatography. Removal of inactive proteins may be carried out using molecular sieve chromatography or ultrafiltration under acidic conditions. For example a molecular sieve chromatography process is used to obtain a composition including metalloproteinase inhibitors having molecular weights of 21,000 Da. In a preferred form, the molecular sieve chromatography resin has a molecular weight range of 10 kDa to 500 kDa. Even more preferably the molecular sieve chromatography resin is Cellufine 1000-M resin having a molecular weight range of 1 0 kDa to 500 kDa.

The suitability of the molecular sieve chromatography resin can be evaluated by passing lactational secretion or derivative thereof through a column of the resin to be tested at neutral pH, or at another defined pH that is advantageous for separation, and measuring the proportion of metalloproteinase inhibitors, and in particular TIMP-2, using a gelatinase activity assay or like assay available in the prior art.

The metalloproteinase inhibitor can be eluted from the column by a suitable eluate buffer as described in the prior art. A suitable eluate buffer includes 150 mM NaCl, 10 mM HCl, pH 2.0.

In a highly preferred form of the invention, the method for preparing a composition including an inhibitor of a metalloproteinase includes treating the lactational secretion or derivative thereof sequentially, first using a filtration step, to remove insoluble materials therefrom; adjusting the pH of the filtrate to between approximately 6.5 to 8.0; contacting the filtrate with a cationic exchange resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay. The major proteins with acidic isoelectric points in the milk product are not absorbed; eluting from the cationic exchange with a suitable buffer solution; subjecting the eluate to a concentration step and diafiltration step to remove salt therefrom; providing a source of acid; subjecting the composition including an inhibitor of a metalloproteinase to transient acidification; contacting the composition with a suitable molecular sieve chromatography resin so that metalloproteinase inhibitors with basic to neutral isoelectric points and even more preferably TIMP-2 with an isoelectric point of 7.0, are enriched in sufficient quantities such that the activity of the metalloproteinase inhibitors are detected using the metalloproteinase inhibitory activity of metalloproteinase 2 and metalloproteinase 9 as detected using a gelatinase activity assay; eluting from the molecular sieve chromatography resin with a suitable buffer solution to obtain a composition further including an inhibitor of a metalloproteinase.

In a preferred process a fraction from the gel filtration chromatography step containing a metalloproteinase inhibitor is subjected to anion exchange chromatography. Suitable anion exchange resins will have the ability to adsorb metalloproteinase inhibitors, preferably metalloproteinase inhibitors with basic to neutral isoelectric points and even more preferably TIMP-2 with an isoelectric point of 7.0, in sufficient quantities such that the activity of the metalloproteinase inhibitors is detected using the metalloproteinase inhibitory activity of metalloproteinase 2 and metalloproteinase 9 as detected using a gelatinase activity assay. Preferably, the anion exchange resin is a Q-Sepharose Fast Flow resin.

The suitability of the anion exchange resin can be evaluated by passing a lactational secretion or derivative thereof through a column of the resin to be tested at neutral pH, or at another defined pH that is advantageous for the adsorption or separation, and measuring the proportion of metalloproteinase inhibitors, and in particular TIMP-2, using a gelatinase activity assay or like assay available in the prior art.

The composition including an inhibitor of a metalloproteinase can be eluted from the resin with a suitable elution buffer. For example a linear salt gradient of 0-0.5M NaCl in 20 mM Tris-HCl can be used.

As a further example, the method for preparing a composition including an inhibitor of a metalloproteinase includes treating a lactational secretion or derivative thereof sequentially by first using a filtration step to remove insoluble materials therefrom; adjusting the pH of the filtrate to between approximately 6.5 to 8.0; contacting the filtrate with a cationic exchange resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay and wherein the major proteins with acidic isoelectric points in the milk product are not absorbed; eluting from the cationic exchange with a suitable buffer solution; subjecting the eluate to a concentration step and diafiltration step to remove salt therefrom; providing a source of acid; subjecting the composition including an inhibitor of a metalloproteinase to transient acidification; contacting the composition with a suitable molecular sieve chromatography resin so that metalloproteinase inhibitors with basic to neutral isoelectric points and even more preferably TIMP-2 with an isoelectric point of 7.0, are enriched in sufficient quantities such that the activity of the metalloproteinase inhibitors are detected using the metalloproteinase inhibitory activity of metalloproteinase 2 and metalloproteinase 9 as detected using a gelatinase activity assay; eluting from the molecular sieve chromatography resin with a suitable buffer solution to obtain a composition further including an inhibitor of a metalloproteinase; contacting the composition with a suitable anion exchange chromatography resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay; eluting from the anion exchange chromatography resin with a suitable buffer solution to obtain a composition including an inhibitor of a metalloproteinase.

In a preferred embodiment a fraction from the anion exchange chromatography step containing a metalloproteinase inhibitor is subjected to affinity chromatography. Preferably, the affinity-exchange column is a heparin-based affinity column.

A suitable affinity chromatography resin will have the ability to adsorb metalloproteinase inhibitors, preferably metalloproteinase inhibitors with basic to neutral isoelectric points and even more preferably TIMP-2 with an isoelectric point of 7.0, in sufficient quantities such that the activity of the metalloproteinase inhibitors is detected using the metalloproteinase inhibitory activity of metalloproteinase 2 and metalloproteinase 9 as detected using a gelatinase activity assay. Preferably, the affinity chromatography resin is a heparin affinity resin, and even more preferably the resin is a Hi-Trap Heparin Sepharose resin (Amersham Pharmacia Biotech).

The suitability of the affinity chromatography resin can be evaluated by passing a milk product through a column of the resin to be tested at neutral pH, or at another defined pH that is advantageous for the adsorption or separation, and measuring the proportion of metalloproteinase inhibitors, and in particular TIMP-2, using a gelatinase activity assay or like assay available in the prior art.

The composition including an inhibitor of a metalloproteinase can be eluted from the resin with a suitable elution buffer. For example a linear salt gradient of 0.1-1.0M NaCl in 20 mM Tris-HCl can be used. For example, the method for preparing a composition including an inhibitor of a metalloproteinase may include treating milk produce sequentially by subjecting the milk product to a filtration step, to remove insoluble materials therefrom; adjusting the pH of the filtrate to between approximately 6.5 to 8.0; contacting the filtrate with a cationic exchange resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay and wherein the major proteins with acidic isoelectric points in the milk product are not absorbed; eluting from the cationic exchange with a suitable buffer solution; subjecting the eluate to a concentration step and diafiltration step to remove salt therefrom; providing a source of acid; subjecting the composition including an inhibitor of a metalloproteinase to transient acidification; contacting the composition with a suitable molecular sieve chromatography resin so that metalloproteinase inhibitors with basic to neutral isoelectric points and even more preferably TIMP-2 with an isoelectric point of 7.0, are enriched in sufficient quantities such that the activity of the metalloproteinase inhibitors are detected using the metalloproteinase inhibitory activity of metalloproteinase 2 and metalloproteinase 9 as detected using a gelatinase activity assay; eluting from the molecular sieve chromatography resin with a suitable buffer solution to obtain a composition including an inhibitor of a metalloproteinase; contacting the composition with a suitable anion exchange chromatography resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay; eluting from the anion exchange chromatography resin with a suitable buffer solution to obtain a composition including an inhibitor of a metalloproteinase; contacting the composition with a suitable affinity chromatography resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay; eluting from the affinity chromatography resin with a suitable buffer solution to obtain a composition including an inhibitor of a metalloproteinase.

Preferably a fraction from the affinity chromatography step is subjected to reverse phase high performance liquid chromatography. A suitable reverse phase high performance liquid chromatography resin is able to adsorb metalloproteinase inhibitors, preferably metalloproteinase inhibitors with basic to neutral isoelectric points and even more preferably TIMP-2 with an isoelectric point of 7.0, in sufficient quantities such that the activity of the metalloproteinase inhibitors is detected using the metalloproteinase inhibitory activity of metalloproteinase 2 and metalloproteinase 9 as detected using a gelatinase activity assay. Preferably, the reverse phase high performance liquid chromatography resin is selected from the group including C4, C8 or C18 matrix. Even more preferably, the reverse phase high performance liquid chromatography resin is a C4 RP-HPLC column.

The suitability of the reverse phase high performance liquid chromatography resin can be evaluated by passing a milk product through a column of the resin to be tested and measuring the proportion of metalloproteinase inhibitors, and in particular TIMP-2, using a gelatinase activity assay or like assay available in the prior art.

The composition including an inhibitor of a metalloproteinase can be eluted from the resin with a suitable elution buffer. For example a 0-28% CH₃CN gradient may be used.

The method for preparing a composition including an inhibitor of a metalloproteinase may include treating milk produce sequentially by subjecting the milk product to a filtration step, to remove insoluble materials therefrom; adjusting the pH of the filtrate to between approximately 6.5 to 8.0; contacting the filtrate with a cationic exchange resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay and wherein the major proteins with acidic isoelectric points in the milk product are not absorbed; eluting from the cationic exchange with a suitable buffer solution; subjecting the eluate to a concentration step and diafiltration step to remove salt therefrom; providing a source of acid; subjecting the composition including an inhibitor of a metalloproteinase to transient acidification; contacting the composition with a suitable molecular sieve chromatography resin so that metalloproteinase inhibitors with basic to neutral isoelectric points and even more preferably TIMP-2 with an isoelectric point of 7.0, are enriched in sufficient quantities such that the activity of the metalloproteinase inhibitors are detected using the metalloproteinase inhibitory activity of metalloproteinase 2 and metalloproteinase 9 as detected using a gelatinase activity assay; eluting from the molecular sieve chromatography resin with a suitable buffer solution to obtain a composition including an inhibitor of a metalloproteinase; contacting the composition with a suitable anion exchange chromatography resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay; eluting from the anion exchange chromatography resin with a suitable buffer solution to obtain a composition including an inhibitor of a metalloproteinase; contacting the composition with a suitable affinity chromatography resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay; eluting from the affinity chromatography resin with a suitable buffer solution to obtain a composition including an inhibitor of a metalloproteinase; contacting the composition with a suitable reverse phase high performance liquid chromatography resin so that metalloproteinase inhibitors with basic to neutral isoelectric points and even more preferably TIMP-2 with an isoelectric point of 7.0, are adsorbed in sufficient quantities such that the activity of the metalloproteinase inhibitors are detected using the metalloproteinase inhibitory activity of metalloproteinase 2 and metalloproteinase 9 as detected using a gelatinase activity assay; eluting from the suitable reverse phase high performance liquid chromatography resin with a suitable buffer solution to obtain a composition further including an inhibitor of a metalloproteinase.

Preferably the treatment step is ultrafiltration. An alternative method for preparing the composition including an inhibitor of a metalloproteinase includes for example, the steps of: providing a source of milk product, a suitable ultrafiltration membrane; contacting the milk product with the suitable ultrafiltration membrane to selectively enrich the filtrate with a property of inhibiting metalloproteinases.

The inventors have surprisingly identified the presence of a metalloproteinase inhibitor in milk products substantially enriched in TIMP-2 polypeptide activity. Based on the teachings of this specification, and as the biological and structural attributes of TIMP-2 has been widely published in the scientific art, the selection of suitable ultrafiltration membranes and use of suitable ultrafiltration to obtain a composition including an inhibitor of a metalloproteinase would require no more than routine experimentation. Examples 1A, 1B, 2 and 3 demonstrate to a skilled artisan the parameters of the physical properties of TIMP-2 and the behavioral properties of TIMP-2 subjected to a variety of environmental conditions. Importantly, the teachings of the specification provide the solubility of TIMP-2 in a variety of buffers and solvents, the charge and isoelectric point of TIMP-2 and the protein size. With these teachings provided by the specification and the high level of knowledge in the art, skilled artisans would readily select and use with a high expectation of success, suitable ultrafiltration membranes in a variety of combinations to purify TIMP-2.

A suitable ultrafiltration membrane will have the ability to enrich a milk product in metalloproteinase inhibitors, preferably metalloproteinase inhibitors with basic to neutral isoelectric points and even more preferably TIMP-2 with an isoelectric point of 7.0, in sufficient quantities such that the activity of the metalloproteinase inhibitors is detected using the metalloproteinase inhibitory activity of metalloproteinase 2 and metalloproteinase 9 as detected using a gelatinase activity assay.

The suitability of the ultrafiltration membrane can be evaluated by passing a milk product through a ultrafiltration membrane at a defined pH that is advantageous for the separation, and measuring the proportion of metalloproteinase inhibitors, and in particular TIMP-2, using a gelatinase activity assay, migration as a single band of approximately 21,000 Da following SDS-PAGE, N-terminal sequence analysis or mass spectrometry or like assay available in the prior art. Preferably, the ultrafiltration membrane has a suitable molecular weight range to separate TIMP-2.

In a preferred embodiment the treatment steps include the subjecting the lactational secretion or derivative thereof to clarification by centrifugation, subjecting the resulting infinite to a suitable affinity chromatography resin such as a heparin affinity resin, and even more preferably the resin is a Hi-Trap Heparin Sepharose resin (Amersham Pharmacia Biotech). The composition including an inhibitor of a metalloproteinase can be eluted from the resin with a suitable elution buffer. For example a linear salt gradient of 0.1-1.0M NaCl in 20 mM Tris-HCl can be used. The eluate can be subjected to a concentration step and diafiltration step to remove salt therefrom, to obtain a composition including an inhibitor of a metalloproteinase.

As used herein the term “comprising” means “including but not limited to”, and that the word “comprises” has a corresponding meaning.

The present invention will now be more fully described with respect to the following examples. Examples 6 to 9 are prophetic examples. It should be understood, however, that the description following is illustrative only, and should not be taken in any way as a restriction on the generality of the invention described above.

### DESCRIPTION OF THE FIGURES

FIG. 1: Bovine cheese whey was subjected to a series of purification steps as exemplified by Example 1A. The figure illustrates the elution profile of protein and metalloproteinase inhibitor in an EIA following each chromatographic step. Protein (−) was estimated by either BCA assay (FIG. 1a-c) or by absorbance at 214 nm (FIG. 1d) (●). Metalloproteinase inhibitor in each fraction was determined by EIA as described in Example 1A.

FIG. 2: SDS-PAGE analysis of purified metalloproteinase inhibitor (left panel) and subsequent western blot (right panel) analysis with an anti-TIMP-2 antibody. Samples of purified metalloproteinase inhibitor loaded at amounts of 2.8, 1.4 or 0.7 μg were run on a 10-20% Tris-tricine gel under reducing conditions and stained with Coomassie Brilliant Blue. Samples of purified metalloproteinase inhibitor loaded at an amount of 0.3 μg was also run on a 10-20% Tris-tricine gel under reducing conditions but was transferred to nitrocellulose and probed with a monoclonal anti-TIMP-2 antibody (Chemicon) and then with HRP-conjugated sheep anti-mouse IgG (Silenus Labs). HRP-labelled proteins were then visualised by enhanced chemoluminescence (ECL). This figure confirms that the metalloproteinase inhibitor is bovine TIMP-2.

FIG. 3: Analytical RP-HPLC analysis of purified metalloproteinase inhibitor. An aliquot (10 ug) of the purified metalloproteinase inhibitor preparation was analysed on a microbore C4 RP-HPLC column (2.1×100 mm, Brownlee Labs). Protein was eluted with an linear gradient of 15-45% CH₃CN in 0.08% TFA over 25 min and then 45-80% CH₃CN over 5 min.

FIG. 4: Mass spectrometry analysis of metalloproteinase inhibitor. The purity and molecular weight of metalloproteinase inhibitor was estimated by mass spectrometry using a VG Quattro triple quadrapole mass spectrometer according to manufacturers instructions. This figure confirms that the metalloproteinase inhibitor is bovine TIMP-2.

FIG. 5: Schematic illustrating the metalloproteinase activity assay used to test the activity of metalloproteinase inhibitor. In this context MMI refers to metalloproteinase inhibitor. This assay utilises a biotinylated gelatinase substrate, which is cleaved by active MMP-2 and MMP-9 (gelatinase) enzymes. The cleavage of the biotinylated gelatinase substrate produces fragments with fewer biotin labels that can bind to a proprietary 96-well plate that is specific for the substrate. The bound fragments are then detected with streptavidin-enzyme complex producing a coloured product upon addition of the enzyme substrate (detectable at 405 nm). Thus, with more gelatinase activity, a lower OD will result, whereas inhibition of gelatinase activity produces a higher absorbance.

FIG. 6: Metalloproteinase inhibitory activity of metalloproteinase inhibitor. Aliquots of pooled fractions from each step in the purification and also fraction 30 and 33 from the cellufine column were analysed for metalloproteinase inhibitory activity using a Gelatinase Activity Assay Kit described above.

FIG. 7: Rate of healing of normal and ischemic wounds at proximal (a), medial (b) and distal (c) sites.

FIG. 8: Western blotting of protein from normal and ischemic wound sites. MMP-9 was upregulated in ischemic wounds at the proximal (P) and medial (M) sites but not at the distal (D) site.

FIG. 9: Western blotting analysis of TIMP-2 in a cellufine fraction.

FIG. 10: Ischemic and normal wound size (% of wound diameter at time 0, at the medial site) after 10 days treatment with saline or Composition A. Values are means±SE. * indicates significant difference by ANOVA to the ischemic wound treated with saline where P<0.05.

### EXAMPLE 1A

Methods for Preparation of Compositions Enriched in Metalloproteinase Inhibitory Activity

Step 1: Cationic Exchange Chromatography

Pasteurized whey obtained as an end product of cheese manufacture was filtered through a 10 micron screen and a 0.2 micron Sartorius Microsart Sartocon II module to remove solids. The ultrafiltrate was adjusted to pH 6.5 and applied to a column of S-Sepharose Fast Flow S cation exchange resin (Pharmacia) that had been equilibrated with 50 mM sodium citrate buffer at pH 6.5. After washing the column with the same buffer the absorbed material was eluted by a solution of 1M NaCl containing 0.25 M NH₄OH. This eluate was diafiltered against water until the conductivity reached 0 Tg and then concentrated by ultrafiltration; both processes using a 3 kDa-excluding membrane. The resultant preparation was freeze-dried for storage.

Step 2: Transient Acidification and Gel Filtration Chromatography (Molecular Sieve Chromatography)

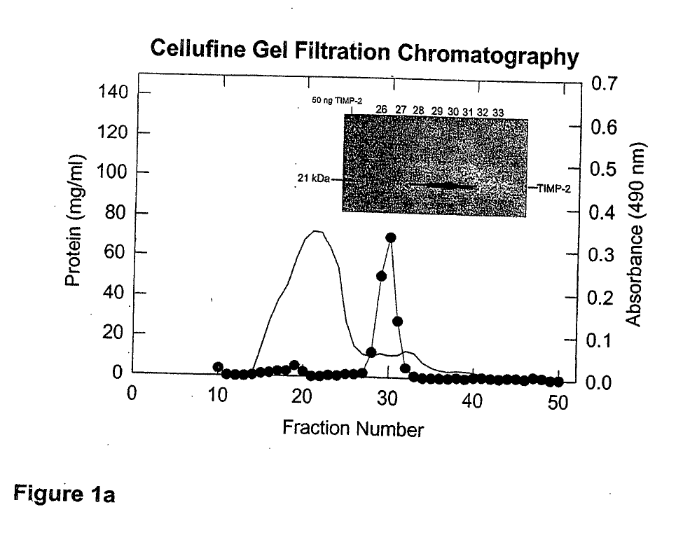

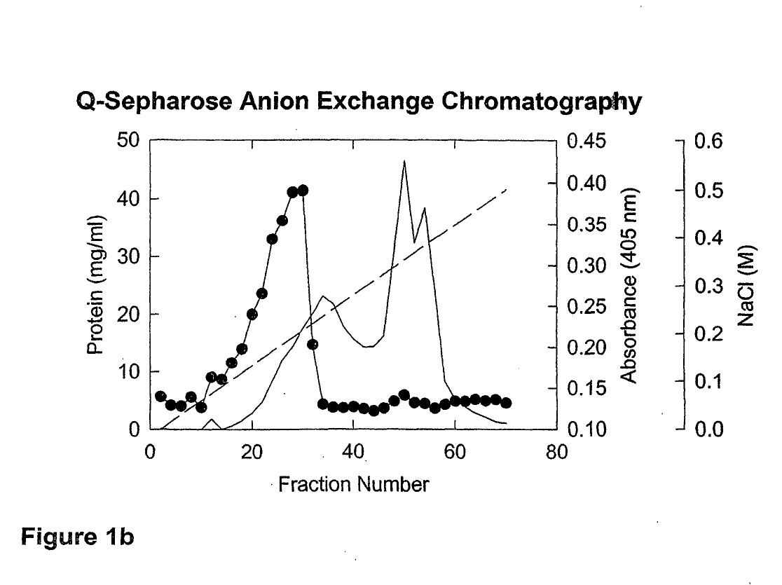

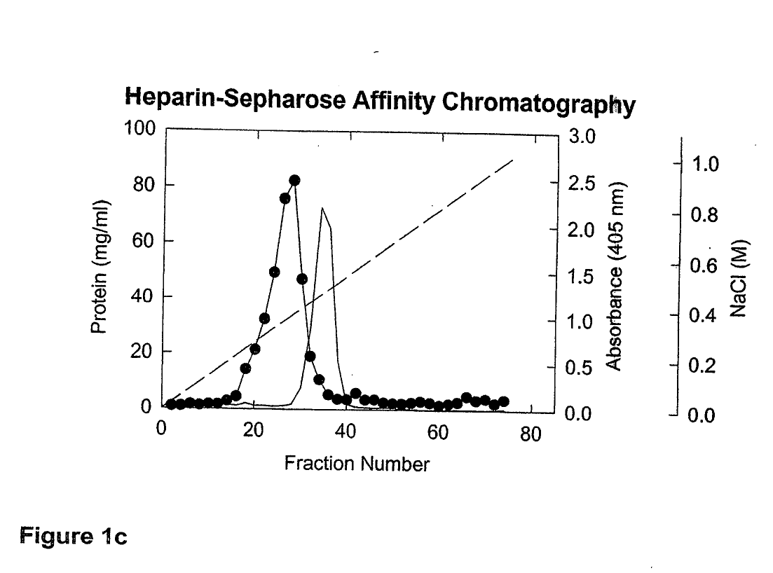

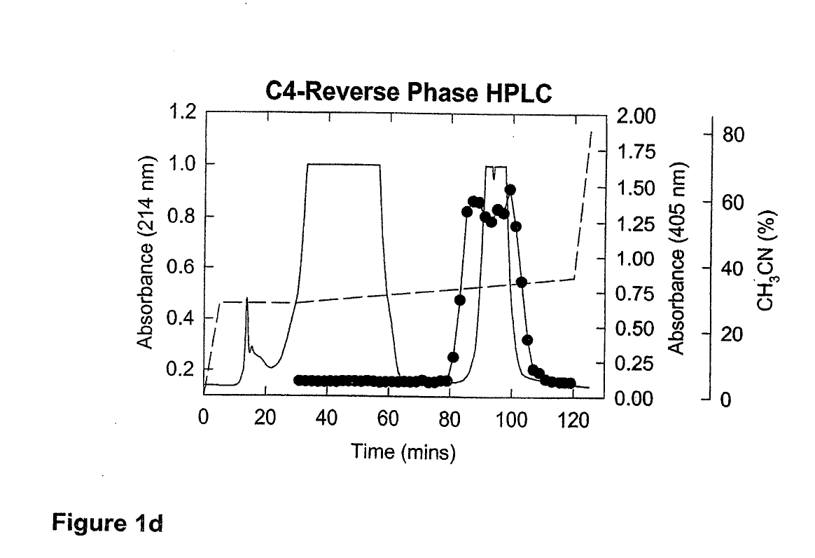

1 L of a composition enriched with a property of inhibiting metalloproteinases as prepared above (total protein concentration=40 mg.mL⁻¹) is acidified to pH 2.0 with HCl overnight and filtered through a 1 μm membrane and applied to two 10 L Cellufine 1000-M columns (molecular weight range of 10-500 kDa) (Amicon) connected in series at a flow rate of 60 mL.min⁻¹. Protein is eluted with 150 mM NaCl, 10 mM HCl, pH 2.0 at a flow rate of 60 mL.min⁻¹. Fractions of 450 mL are collected and analysed for protein concentration (BCA assay) and metalloproteinase inhibiting activity with an enzyme immunoassay (EIA) (see below) (FIG. 1a). An aliquot of each fraction (20 μg) enriched with a property of inhibiting metalloproteinases is also run on a 4-20% reducing Tris-Glycine gel, transferred to nitrocellulose and blotted for metalloproteinase inhibiting activity with an monoclonal anti-TIMP-2 antibody to confirm the presence of bovine TIMP-2 at the correct molecular weight in the EIA-positive fractions (FIG. 1a, inset)

Step 3: Anion Exchange Chromatography

The pooled cellufine fractions are made to 50 mM NaCl, 20 mM Tris-HCl, pH 8.0 with solid base Tris, and pH adjustment to 8.0 with 5 M HCl, filtered through a 1 μm membrane, and applied to a Q-Sepharose Fast Flow column (5×15 cm; Pharmacia, Sweden) attached to an FPLC system (Pharmacia, Sweden) at a flow rate of 5 ml.min⁻¹. The column is then washed with 20 mM Tris-HCl and the proteins that remain bound to the column (which include metalloproteinase inhibitor) are eluted with a 2.1 L linear salt gradient of 0-0.5 M NaCl in 20 mM Tris-HCl at a flow rate of 15 mL.min⁻¹. Fractions of 30 mL are collected and analysed for protein concentration and metalloproteinase inhibitor (FIG. 1b). Fractions containing metalloproteinase inhibiting activity are then pooled.

Step 4: Affinity Chromatography

The pooled Q-Sepharose Fast Flow fractions are applied to a Hi-Trap Heparin Sepharose column (two 5 mL columns in series; Pharmacia, Sweden) attached to an FPLC system (Pharmacia, Sweden) at a flow rate of 2 mL.min⁻¹. The column is then washed with 0.1 M NaCl, 20 mM Tris-HCl, pH 8.0 and the proteins that remain bound to the column (which include metalloproteinase inhibiting fractions) are eluted with a 75 mL linear salt gradient of 0.1-1.0 M NaCl in 20 mM Tris-HCl at a flow rate of 15 mL.min⁻¹. Fractions of 1.0 mL are collected and analysed for protein concentration and metalloproteinase inhibiting activity (FIG. 1c). Fractions containing metalloproteinase inhibiting activity are then pooled.

Step 5: C4 RP-HPLC

The pooled Hi-Trap Heparin Sepharose fractions are diluted 1:2 with 0.1% TFA and applied to a Delta-Pack C4 RP-HPLC column (15 μm, 300 Å, 25×100 mm, Millipore-Waters, Lane Cove, New South Wales, Australia) equilibrated with 0.1% TFA. The column is washed extensively with 0.1% TFA, then bound protein eluted successively with a 0-28% CH3CN gradient over 25 min and then a gradient of 28-36% CH₃CN in 0.08% TFA over 90 min at a flow rate of 5 mL.min⁻¹. Fractions of 5 mL are collected and assayed for metalloproteinase inhibiting activity (FIG. 1d). Fractions containing metalloproteinase inhibiting activity are pooled and lyophilised.

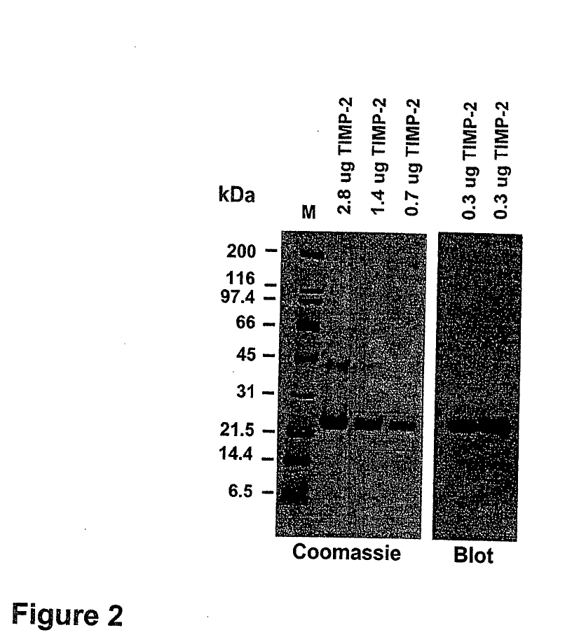

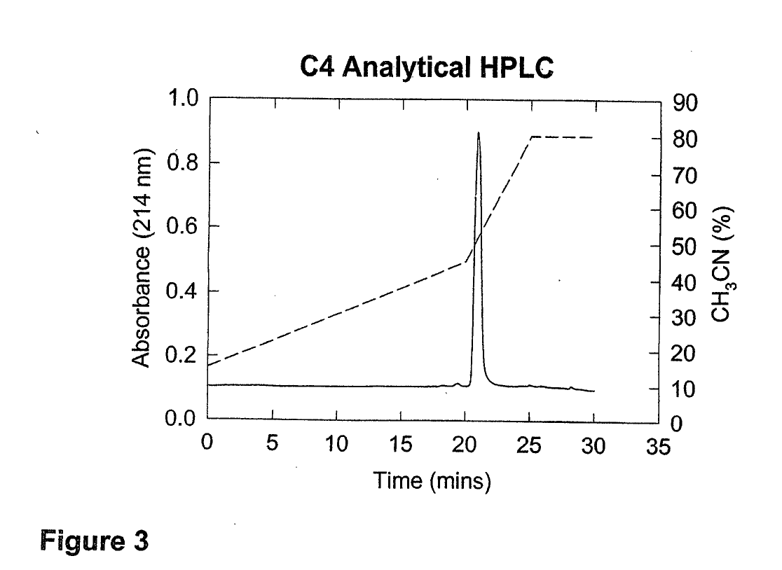

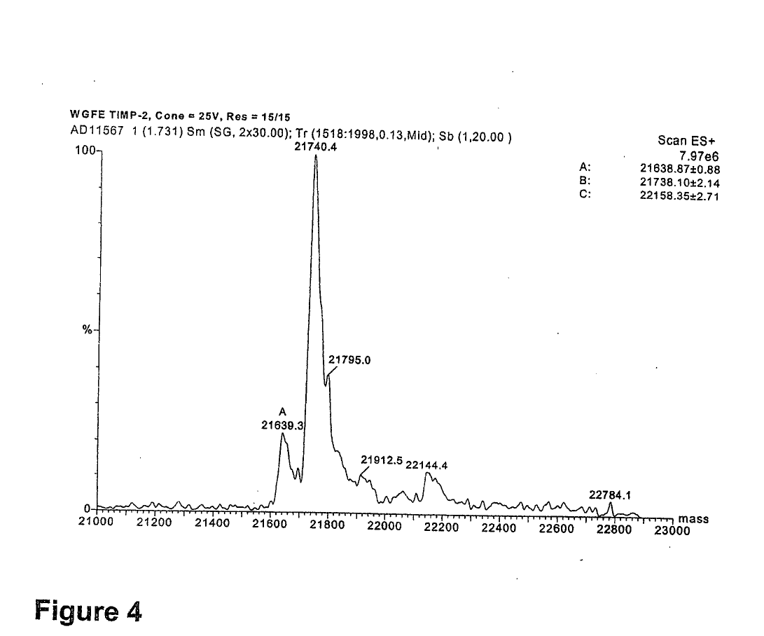

The metalloproteinase inhibiting activity is present in the positive fractions from the final RP-HPLC step in a form which is up to, and including, 99% pure bovine TIMP-2 and has a molecular weight of approximately 21 kDa. These criteria being estimated from Coomassie stained reducing 10-20% gradient Tricine SDS-PAGE gel (see FIG. 2), analytical RP-HPLC (FIG. 3), mass spectrometry analysis (FIG. 4) and N-terminal sequence analysis.

TIMP-2 Enzyme Immunoassay

Individual wells of a 96-well immunoplate (Nunc) are coated with 90 μL of each sample (column fractions) in 15 mM Na₂CO₃, 35 mM NaHCO₃, pH 9.6 at 4° C. overnight. The plates are then washed 4 times with 100 μl 0.05% Tween-20 in PBS and blocked for 1 hr at 37° C. in 1% BSA in PBS. Plates are washed 4 times with 0.05% Tween-20 in PBS and incubated with 1 μg.mL⁻¹ mouse anti-human TIMP-2 monoclonal IgG for 1 hr at 37° C. (Chemicon). Plates are washed again 4 times with 100 μl 0.05% Tween-20 in PBS and incubated with sheep anti-mouse HRP conjugated IgG (diluted 1:10000) for 1 hr at 37° C. Plates are then washed again 4 times with 0.05% Tween-20 in PBS and developed with 200 μL per well of 2,2′-Azino-bis(3-ethylbenzthiazoline-6-sulfonic acid) (ABTS) substrate for 1 hr at 37° C. and absorbance determined at 405 nm. In some cases the EIA was developed with 200 μL o-Phenylenediamine dihydrochloride (OPD) and absorbance determined at 490 nm.

### EXAMPLE 1B

Alternative Methods for Preparation of Compositions Enriched in Metalloproteinase Inhibitory Activity

A volume of reconstituted skim milk powder, or defatted Colostrum is clarified by centrifugation at 41,000×g for 30 min at 4° C. The clarified feed stock is loaded onto a HiPrep 16/10 Heparin FF column (16×100 mm, Pharmacia, Sweden) attached to an ÄKTA FPLC system (Pharmacia, Sweden) at a flow rate of 5 mL.min⁻¹. The column is washed with 10 mM sodium phosphate buffer, pH 7.0, and the proteins that remain bound to the column (which include metalloproteinase inhibiting fractions) are eluted with 10 mM sodium phosphate buffer, pH 7.0, containing 2M NaCl at a flow rate of 5 mL.min⁻¹. The salt is removed from the bound material by loading the eluted material onto a HiPrep Desalting column (Pharmacia, Sweden) and concentrated in an Amicon ultrafiltration cell using a 5,000 nominal molecular weight Biomax membrane (Millipore Corporation, Bedford, Mass.). The composition is analysed for protein concentration and metalloproteinase inhibiting activity quantified by the method in Example 3 (Table 2(b)).

### EXAMPLE 2

N-Terminal Sequence Analysis of Metalloproteinase Inhibitor

The amino acid sequence and hence the identity of metalloproteinase inhibitor was determined by N-terminal sequence analysis. Twenty μg of purified metalloproteinase inhibitor was resuspended in 50 mM Tris-Cl pH 8.5, 6 M guanidine-HCl, 10 mM dithiothreitol (DTT) and incubated at 65° C. for 30 min. The denatured and reduced sample was then cooled to room temperature and alkylated by the addition of 40 mM iodoacetamide and incubation for 30 min. The sample was then acidified by the addition of trifluoacetic acid (TFA) and sequenced from the N-terminus by Edman degradation using a Hewlett-Packard G1000A protein sequencer according to manufacturers instructions. The N-terminal sequence (eight cycles) was:
CSCSPVHP

### EXAMPLE 3

Metalloproteinase Inhibitory Activity of a Composition

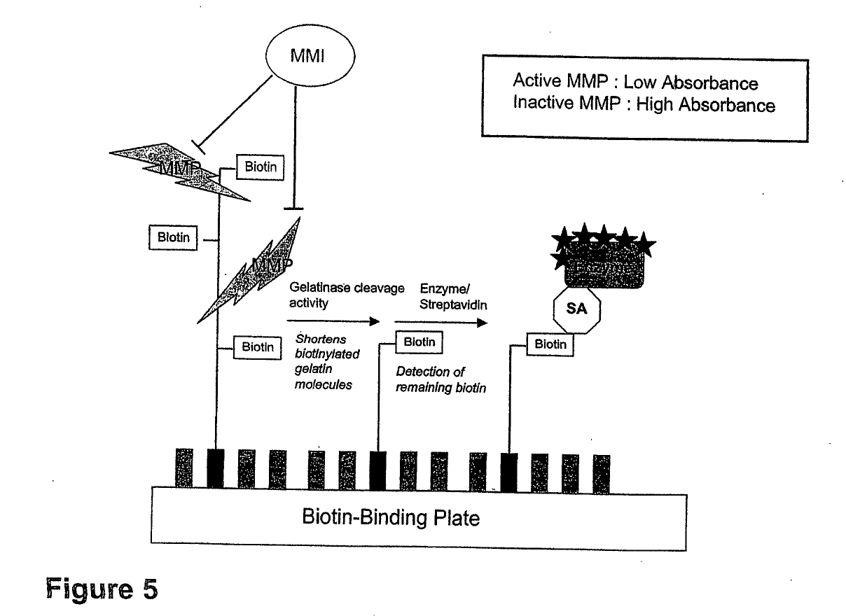

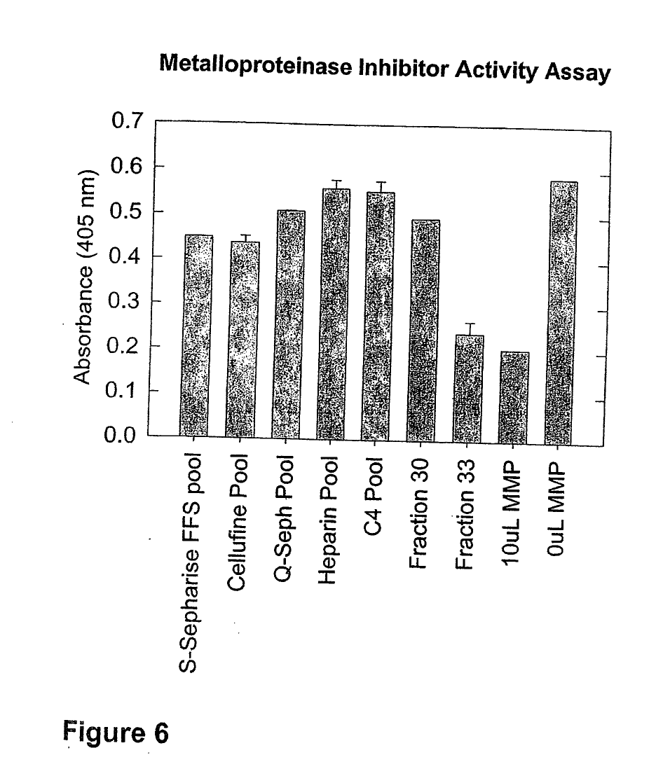

The activity of the metalloproteinase inhibitor was examined using a metallorproteinase Gelatinase Activity Assay Kit (Chemicon). This assay utilises a biotinylated gelatinase substrate, which is cleaved by active MMP-2 and MMP-9 (gelatinase) enzymes. The cleavage of the biotinylated gelatinase substrate produces fragments (with fewer biotin labels that can bind to a proprietary 96-well plate that is specific for the substrate. The bound fragments can then be detected with streptavidin-enzyme complex, which produces a coloured product upon addition of the enzyme substrate (detectable at 405 nm). Thus, with more gelatinase activity, a lower OD will result, whereas inhibition of gelatinase activity produces a higher absorbance (FIG. 5). p-aminophenylmercuric acetate (p-APMA) activated MMP-2 (6.7 ng) in TNC buffer (50 mM Tris-Cl pH 7.5, 0.15 M NaCl, 10 mM CaCl2, 0.05% Brij-35) is pre-incubated either alone or with pooled fractions (10 μl) from each step of the purification for 30 min at 37° C. Subsequently biotinylated gelatinase substrate is added to each sample and incubated a further 30 min at 37° C. and then MMP activity determined according to manufacturers instructions. As shown in FIG. 6, in the absence of MMP-2, the gelatinase substrate remains uncleaved (high absorbance) whereas in the presence of MMP-2 the substrate is cleaved and a low absorbance is observed. Pre-incubation of MMP-2 with metalloproteinase inhibitor-positive fractions from each step in the purification clearly show the inhibitory effect of metalloproteinase inhibitor on metalloproteinase activity. As expected, fraction 33 from the cellufine column, which contains no metalloproteinase inhibitor, (see FIG. 1) did not inhibit metalloproteinase activity whereas fraction 30 containing metalloproteinase inhibitor was successful.

The metalloproteinase inhibitor activity of the milk extract composition from various fraction pools of the purification method outlined in Examples 1A and 1B was also quantified using a fluorescence-quenching substrate by metalloproteinases (Peptide Institute, Osaka, Japan). Cleavage of this substrate by metalloproteinases releases the quencher molecule from the proximity of the fluorescent tag, producing an increase in fluorescence. Thus, an increase in the amount of metalloproteinase inhibitors will result in a decrease in fluorescence. 1 mM p-aminophenylmercuric acid (p-APMA) was used to activate 1.5 μg/mL recombinant human MMP-2 for 1 hour at 37° C. Pooled fractions to be assayed were typically diluted 1:300 with Buffer E (0.1M Tris, 0.1 M NaCl, 0.1 M CaCl₂, 0.0% Brij-35) and a standard curve was generated using 0 to 0.15 μg/mL recombinant human TIMP-2 (R & D Systems, Minneapolis, USA) in Buffer E 40 μL activated MMP-2 and 40 μL of each sample was then plated in individual wells of 96-well trays, which was brought up to a total volume of 270 μL/well with Buffer E. These samples were then incubated for 1 hour at RT, after which, 30 μL/well 16 μM fluorescent substrate was added and incubated for 30-45 min at RT. The reaction was stopped with 30 μL 200 mM EDTA and fluorescence was measured (excitation/emission λ=340-390 nm) on a Wallac 1420 multilable counter (Perkin Elmer, Finland). Results from this experiment are represented in Table 2 below (“MMI” refers to metalloproteinase inhibitor, later confirmed as TIMP-2).

TABLE 2
Metalloproteinase Inhibitor Purification Table
(a) Data from Example 1A

| | Total | | | | | |
|---|---|---|---|---|---|---|
| | protein | MMI | MMI | % | Purification | Yield |
| | (mg) | (ug/mL) | (mg) | purity | (fold) | (%) |
| S-Sepharose Fast Flow | 39291.9 | 45.4 | 45.4 | 0.1 | 1.0 | 100.0 |
| S Pool | | | | | | |
| Cellufine pool | 2462.7 | 15.9 | 39.0 | 1.6 | 13.7 | 85.9 |
| Q-Sepharose pool | 172.5 | 60.2 | 27.1 | 15.7 | 136.0 | 59.7 |
| Heparin pool | 56.7 | 256.2 | 13.7 | 23.0 | 199.4 | 28.8 |
| C4 RP-PLC pool | 10.5 | 50.3 | 10.1 | 95.5 | 826.6 | 22.2 |

(b) Data from Example 1B

| | | Total | Total protein | | MMI | MMI |
|---|---|---|---|---|---|---|
| | Total | protein | Flow | MMI | (ng/mg) | (ng/mg) |
| | protein | bound | Through | (ng/mg) | Flow | Total |
| | (mg/ml) | (mg/ml) | (mg/ml) | Bound | Through | Protein |
| Milk | 18.23 | — | — | — | — | Not |
| | | | | | | detected |
| Milk/Heparin | — | 3.60 | 7.88 | 35.13 | Not | 11.48 |
| | | | | | detected | |
| Colostrum | 30.72 | | | — | — | 8.1 |
| Colostrum/Heparin | — | 9.32 | 14.09 | 66.77 | 0.17 | 27.96 |
| Cheese whey | 8.07 | — | — | | | 4.37 |
| Cheese | 31 | — | — | — | — | 864.12 |
| whey/Cationic | | | | | | |
| exchange fraction | | | | | | |

### EXAMPLE 4

Suitable Pharmaceutical or Veterinary Compositions Enriched in Metalloproteinase Inhibitory Activity

All units for ingredients of the compositions are measured in “parts”.

Milk product extract quantity specified hereby referred to as “qs”.

(i) Saline Solution

| Active substance metalloproteinase inhibitor | qs |
|---|---|
| Sodium Chloride | 0.9 |
| Distilled water to | 100 |

After mixing the components thoroughly, the composition is then pH adjusted to between 3.8-4.2 with the addition of 10 mM HCl.

(ii) Cetomacrogol Cream

| Active substance metalloproteinase inhibitor | qs |
|---|---|
| Cetomacrogol emulsifying wax | 15 |
| Liquid paraffin (by weight) | 10 |
| Chlorocresol | 0.1 |
| Propylene glycol | 5 |
| Distilled water to | 100 |

Cetomacrogol emulsifying wax is melted with paraffin at about 70° C. Chlorocresol and propylene glycol is dissolved in about 50 parts of the distilled water warmed to about the same temperature. After mixing, the composition is adjusted to weight and stirred until cool. Milk product extract is then added to an appropriate concentration, and mixed thoroughly.

(iii) Aqueous Cream APF

| Active substance metalloproteinase inhibitor | qs |
|---|---|
| Emulsifying ointment | 30 |
| Glycerol | 5 |
| Phenoxyethanol | 1 |
| Distilled water to | 100 |

The emulsifying ointment is melted at about 70° C. The phenoxyethanol is dissolved in the distilled water, warmed to about the same temperature. The composition is mixed, adjusted to weight and stirred until cool. The milk product extract is then added while stirring thoroughly.

(iv) Buffered Cream BPC 73

| Active substance metalloproteinase inhibitor | qs |
|---|---|
| Citric acid | 5 |
| Sodium phosphate | 25 |
| Chlorocresol | 1 |
| Emulsifying ointment | 300 |
| Distilled water | 669 |

Emulsifying ointment is melted with the aid of gentle heat, followed by addition of sodium phosphate, citric acid and chlorocresol, previously dissolved in the distilled water at the same temperature. The composition is stirred gently until cold. The milk product extract is then added and mixed thoroughly.

(v) Emulsifying Ointment APF

| Active substance metalloproteinase inhibitor | qs |
|---|---|
| Emulsifying wax | 30 |
| White soft paraffin | 50 |
| Liquid paraffin (by weight) | 20 |

The waxes and paraffins are melted together and stirred until cool. Milk product extract is then added to an appropriate concentration in a portion of the base, gradually incorporating the remainder, followed by thorough mixing.

(vi) Peptide Ointment (as in Neomycin and Bacitracin Ointment BPC 73)

| Active substance metalloproteinase inhibitor | qs |
|---|---|
| Liquid paraffin | 10 |
| White soft paraffin to | 100 |

White soft paraffin is melted, and the liquid paraffin incorporated. The mixture is stirred until cold. The milk product extract is titrated with a portion of the base and gradually incorporated into the remainder of the base.

(vii) Gel (as Used in Lignocaine and Chlorhexidine Gel APF)

| Active substance metalloproteinase inhibitor | qs |
|---|---|
| Tragacanth | 2.5 |
| Glycerol | 25 |
| Distilled water to | 100 |

The tragacanth is mixed with glycerol and most of the distilled water. After bringing to the boil, the mixture is cooled, and the milk product extract is added. The composition is adjusted to weight and mixed well. The finished product is protected from light.

(viii) Spray (as used in Adrenaline and Atropine Spray BPC 73)

| Active substance metalloproteinase inhibitor | qs |
|---|---|
| Sodium metabisulphite | 1 |
| Chlorbutol | 5 |
| Prophylene glycol | 50 |
| Distilled water to | 1000 |

(ix) Spray (as Used in Indospray)

| Active substance metalloproteinase inhibitor | qs |
|---|---|
| Alcohol | 95% |

(x) Lotions (as Used in Aminobenzoic Acid Lotion BPC 73)

| Active substance metalloproteinase inhibitor | qs |
|---|---|
| Glycerol | 20 |
| Alcohol 95% | 60 |
| Distilled water to | 100 |

(xi) Cetomacrogol Lotion APF

| Active substance metalloproteinase inhibitor | qs |
|---|---|
| Cetomacrogol emulsifying wax 3 | |
| Liquid paraffin | 10 |
| Glycerol | 10 |
| Chlorhexidine gluconate solution | 0.1 |
| Distilled water to | 100 |

Cetomacrogol emulsifying wax is melted with the liquid paraffin at about 60° C. To this mixture, the chlorhexidine solution previously diluted to 50 parts is added, with rapid stirring, with distilled water at the same temperature. After mixing, the composition is adjusted to volume and stirred until cold.

(xii) Mouthwash

| Active substance metalloproteinase inhibitor | qs |
|---|---|
| Polyethylene glycol (7)-glycerol cocoate | 2 |
| Glycerin (86%) | 18 |
| Peppermint oil | 10 |
| Ethanol | 55 |
| Distilled water | 13 |

Dissolve the active ingredients in distilled water. Add and dissolve polyethylene glycol (7)-glycerol cocoate and glycerin in the solution. Dissolve peppermint oil in ethanol and mix the two solutions with stirring. The solution is diluted up to 1:10 before use.

(xiii) Toothpaste

| Active substance metalloproteinase inhibitor | qs |
|---|---|
| Methyl cellulose | 0.8 |
| Calcium carbonate | 30 |
| Colloidal silica | 3 |
| Light liquid paraffin | 2 |
| Glycerin | 20 |
| Distilled water | 100 |

The powders are wetted with the mixture of the active substances and methyl cellulose in a part of distilled water, paraffin and glycerin. After making up with the remaining water the paste is homogenized.

(xiv) Parenteral Solution Ingredients

| Active substance metalloproteinase inhibitor | qs |
|---|---|
| +/− Human serum albumin | 2 |
| Arginine or glycine | 40 |
| +/− Carbohydrate | 40 |

The carbohydrate is glucose, mannose, dextran, hydroxyethyl starch or a mixture thereof. The pH is adjusted with phosphate, succinate, amino acids or a mixture thereof.

### EXAMPLE 5

Effectiveness of a Composition Derived from a Milk Product Being Enriched with a Property of Inhibiting Metalloproteinases as a Therapeutic Treatment for Chronic Wounds

The invention may be used to prevent and treat damage relating to surface wounds.

Metalloproteinases are not typically expressed in normal tissues but are up-regulated when repair or remodeling of tissue is required and in ulcerations and chronically inflamed tissue. A chronic wound is an open wound involving tissue loss which persists for long periods. Burns are considered to be chronic wounds, as are dermal or skin ulceration caused from pressure and internal circulatory problems which cause inadequate blood flow and tissue death. These wounds are characterised by an excess of matrix metalloproteinases that prevent effective wound healing and closure. Metalloproteinase inhibitors and in particular, TIMP-2 inhibits these matrix metalloproteinases. To successfully test the effectiveness of TIMP-2 in assisting wound healing, a suitable animal model is required that reflects the characteristics of chronic wounds found in human patients. Typical characteristics are as follows;
1) Chronic wounds have higher levels of MMP-9 and to a lesser extent MMP-2 than normal wounds determined by zymogram analysis (Trengrove et al., Wound Repair Regen. 1999 Nov-Dec; 7(6):442-52);
2) MMP-1 (collagenase 1) and MMP-9 mRNA levels increase soon after wounding and is followed by an increase in MMP-2 mRNA expression (Soo et al. Plast Reconstr Surg. 2000 February; 105(2):638-47);
3) MMP-2 and MMP-9 levels are increased 5-10 fold in fluid from chronic leg ulcers (Wysocki et al, J Invest Dermatol. 1993 July; 101(1):64-8);
4) In chronic wounds zymograms demonstrate increased gelatinase activity (MMP-2 and MMP-9) that includes lower molecular weight proteinase species that may represent activated or superactivated gelatinase fragments (Bullen et al, J Invest Dermatol. 1995 February; 104(2):236-40).

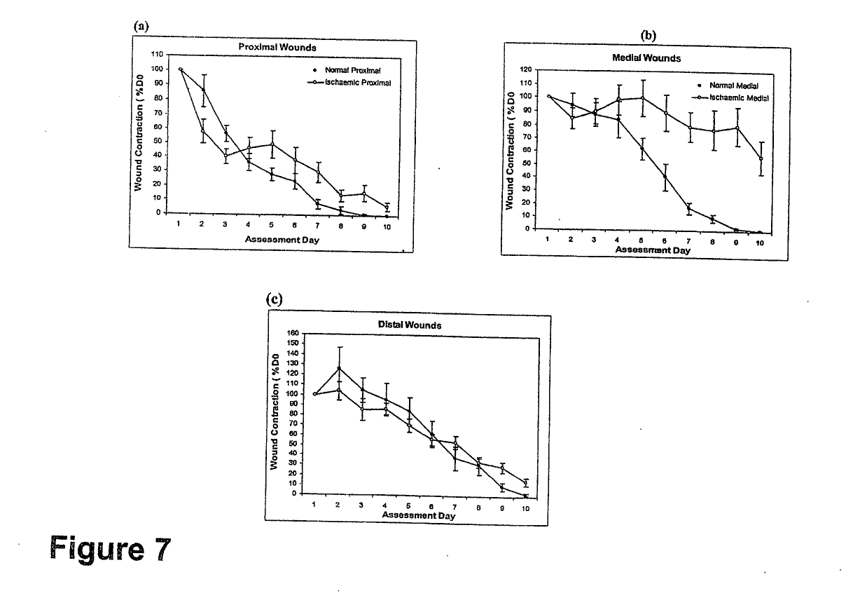

The rat model of chronic wounds developed by Chen et al (1999) was used to demonstrate the effectiveness of milk product enriched with a property of inhibiting metalloproteinases as a therapeutic treatment for chronic wounds. In this model, full thickness wounds were created in the backs of male Sprague-Dawley rats using a 6 mm skin biopsy punch in an electric drill (normal wound). Pairs of wounds were created at proximal, medial and distal sites. To create ischemic wounds two parallel incisions were made along the sides of the wounds and the skin flap elevated, repositioned and sutured. The healing of ischemic wounds at the medial site was significantly slower than that of normal wounds (FIG. 7b). In contrast, healing rates at the proximal and distal sites were significantly faster to that seen at the medial sites, with the ischemic effect least apparent at the distal sites (FIGS. 7a & 7c). The differential healing rate is most likely a reflection of differential blood flow to the various wound sites and an overall increase in MMP levels, characterised mainly by an increase in proMMP-9 and other unidentified gelatin degrading proteinases.

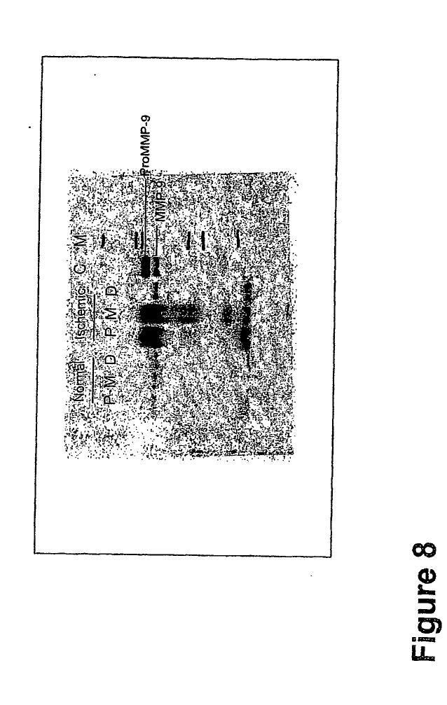

Western immunoblot analysis was conducted on pooled samples of normal (n=3-5) and ischemic (N=5-6) wounds at each wound site (FIG. 8). Levels of MMP-9 were dramatically increased in the ischemic wounds at the proximal and medial sites but not the distal site, reflecting the pattern seen with the wound healing rate. Ischemic wounds demonstrated significantly slower healing rates than normal wounds and MMP-9 was upregulated in the proximal and medial ischemic wounds. This evidence suggests that the rat ischemic wounds show characteristics typical of chronic wounds found in human patients.

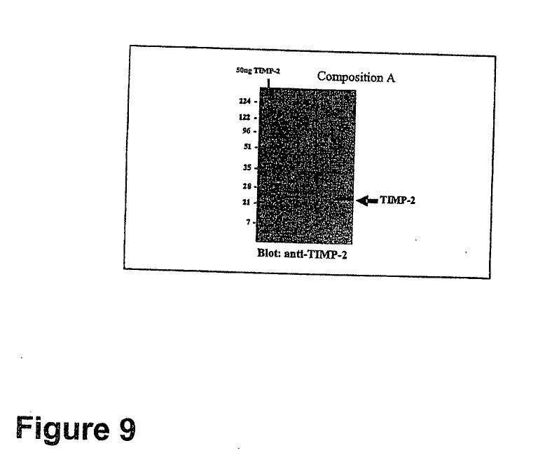

Composition A, enriched in metalloproteinase inhibitory activity was obtained from whey which had been subjected to steps 1 and 2 as outlined in Example 1A. The biologically active TIMP-2 component of Composition A was quantified using a purified TIMP-2 standard curve in a fluorogenic MMP inhibition as outlined in Example 1A. Composition A contained 10.82 μg/ml TIMP-2. Western immunoblot analysis of Composition A confirmed that the TIMP-2 reactivity was the 22 kDa form of the TIMP-2 protein (FIG. 9).

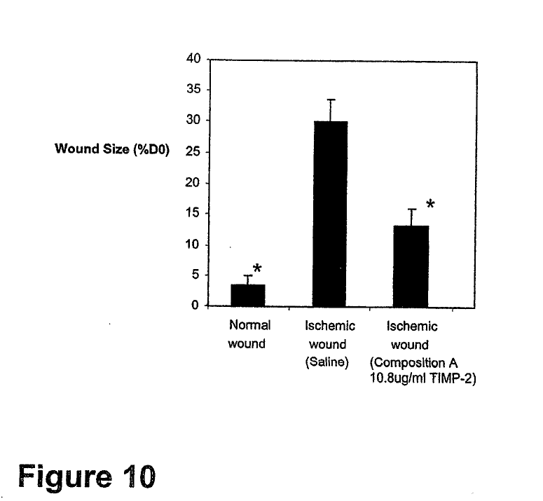

Composition A was tested in the rat chronic wound model for its ability to accelerate ischemic wound healing. The trial was conducted over a 10 day period with 8 rats per treatment group. Each day following surgery, wound size was measured by tracing around the wound on an overhead transparency. Rats were then administered with a dose (150 μl/wound) of the neat Composition A delivered in an alginate dressing. Ischemic control rats were administered sterile saline. After 10 days, rats treated with Composition A showed significantly improved healing when compared to the saline treated controls (FIG. 10).

On the basis of the results shown in Example 5 showing the use of metalloproteinase inhibitors including TIMP-2 to enhance wound healing in the chronic wound, the inventors expect that the invention used in this particular model would decrease MMP-9 activity measured by zymogram analysis for MMP-9 (92 kDa) activity, resulting in an increase in the rate of wound contraction and reduced time to functional recovery of the skin.

### EXAMPLE 6

Prophetic Example Demonstrating Methodologies to Study the Effectiveness of a Composition Enriched with a Property of Inhibiting Metalloproteinases as a Therapeutic Treatment for Oral Mucositis.

The invention may be used to prevent and treat damage relating to mucositis. The person skilled in the art will readily be able to investigate the claimed invention to treat mucositis.

For example, investigations into the effects of a composition enriched with a property of inhibiting metalloproteinases administered topically on chemotherapy-induced mucositis in male Golden Syrian hamsters may be used. Typically the trial would involve the continuous application of compositions enriched with a property of inhibiting metalloproteinases to the cheek pouch of 10 hamsters treated with 5-fluorouracil.

Hamsters with similar mean body weights will then be divided into two groups of five animals. All hamsters will be given intraperitoneal injections of 90 mg/kg of 5-fluorouracil on day 1 and 60 mg/kg on day 3. The cheek patch will be scratched on days 1, 2 and 3 with six strokes of a wire brush in one direction and six strokes in the other perpendicular direction to achieve a uniform wound.

Groups will be treated with either a commercial mouthwash as vehicle, or 0.3 ml of composition enriched with a property of inhibiting metalloproteinases at a specified protein concentration. The cheek pouch liquid treatments will be applied daily for one minute, during which time the hamsters are anaesthetized using isoflurane anesthesia. The cheek pouch will be be assessed on days 5, 7, 8, 11, 13 and 15. Monitoring will be based on a visual assessment of the cheek pouch (graded on a 1-10 scale) taking into account the overall severity of the lesion, degree of bruising, swelling and scarring. Body weight will be be recorded as a percentage of the day 0 value.

On the basis of the results shown in Examples 1A, 1B, 2, 3 and in particular the results shown in Example 5 showing the use of metalloproteinase inhibitors to treat wounds, the inventors expect that the invention used in this particular model would reduce mucositis compared to the vehicle treated group as measured by overall visual score, total ulcer area and body weight loss.

### EXAMPLE 7

Prophetic Example to Demonstrate Methodologies to Study the Effectiveness of a Composition Enriched with a Property of Inhibiting Metalloproteinases to Reduce Bacterial Translocation Across the Gut.

The invention may be used to reduce the bacterial translation across the gut. The person skilled in the art will readily be able to investigate the claimed invention to reduce the bacterial translation across the gut.

The ability of the gut epithelium to provide a barrier against bacterial invasion provides a suitable measure of mucosal repair and subsequently, gut function.

For example, male Sprague Dawley rats injected with high doses of chemotherapy agent, methotrexate will be used as an experimental model of damage to the lining of the alimentary tract. Control rats will receive no composition enriched with a property of inhibiting metalloproteinases whereas experimental rats will be treated for 5 days with a composition enriched with a property of inhibiting metalloproteinases. Treated rats will be fed a modified diet of specified amounts of a composition enriched with a property of inhibiting metalloproteinases in place of the equivalent amount of casein. In addition, treated rats will be given a composition by stomach gavage on days 3, 4 and 5 of the experimental period. Control rats will be fed the unmodified diet and gavaged by an identical protocol on days 3, 4 and 5 with an equivalent amount of bovine serum albumin to ensure an isonitrogenous diet.

One group of control rats and the treated rats will be injected subcutaneously with 2.5 mg/kg methotrexate at the start of days 1, 2 and 3. An additional control group will receive sham methotrexate injections, and will be pair-fed to the methotrexate-injected control group.

Rats will be maintained in the metabolism cages until exsanguinations on days 5, 8 and 12. The abdominal skin will be soaked in 70% ethanol before the intestine is removed under aseptic conditions. All visible mesenteric lymph nodes will be placed into a sterile pre-weighed container and samples then weighed and infusion solution will be added to a final concentration of 100 mg/ml. Tissues are homogenized in this solution with sterile glass-reinforced grinders. For measurement of translocation of gram negative bacteria into mesenteric lymph nodes, 40 or 60 mg of each tissue homogenate will be placed on MacConkey agar or blood agar plates and incubated aerobically at 35° C. for 48 hours. Enteric gram negative bacterial colonies will then be identified using API 20E strips, then counted. The incidence (proportion of animals exhibiting detectable bacterial translocation) and mean number of bacterial colonies per gram of tissue will be calculated for each treatment group.

On the basis of the results shown in Examples 1A, 1B, 2, 3 and in particular the results shown in Example 5 showing the use of metalloproteinase inhibitors to treat wounds, the inventors expect that the invention used in this particular model would result in a lower incidence of translocation. The inventors also expect the number of colonies per gram of mesenteric lymph node will be significantly lower in treated groups.

### EXAMPLE 8

Prophetic Example to Demonstrate Methodologies to Study the Effectiveness of a Composition Enriched with a Property of Inhibiting Metalloproteinases to Prevent Loss of Small Intestinal Crypts and Villi in Rats with Methotrexate-Induced Small Bowel Mucositis.

The invention may be used to prevent loss of small intestinal crypts and villi in rats with methotrexate-induced small bowel mucositis. The person skilled in the art will readily be able to investigate the claimed invention to prevent loss of small intestinal crypts and villi in rats with methotrexate-induced small bowel mucositis.

For example, male Sprague Dawley rats will be injected with high doses of the chemotherapy agent, methotrexate, as an experimental model of gut mucositis. In rats, methotrexate damages the small bowel, but not the oral or colonic mucosa (Vanderhoof J A, Park J H Y, Mohammapour H, Blackwood. Gastroenterology. 1990. 98:1226-1231).

Rats, weighing on average 140 g will be maintained in metabolism cages and fed a high carbohydrate diet. Control rats will receive no composition having metalloproteinase activity, whereas treated rats will be treated for 5 days with a composition having a metalloproteinase inhibitor. In addition, treated rats will be given a composition having a metalloproteinase inhibitor by stomach gavage on days 3, 4 and 5 of the experimental period. Control rats will be fed the unmodified diet and gavaged by an identical protocol on days 3, 4 and 5 with an equivalent amount of bovine serum albumin to ensure an isonitrogenous diet.

One group of control rats and the test rats will be injected subcutaneously with 2.5 mg/kg methotrexate at the start of days 1, 2 and 3. An additional control group will receive sham methotrexate injections, and was pair-fed to the methotrexate-injected control group. Rats will be maintained in the metabolism cages for 5 days, at which time they are killed for collection of the gastrointestinal tract. Tissue samples will then be collected from the proximal small bowel, as well as the distal small bowel. Tissue samples will be fixed and stained for histological analysis using methods described in Read et al., J Endocrinol. 1992 133:421-431, the entire disclosure is incorporated herein by reference.

On the basis of the results shown in Examples 1A, 1B, 2, 3 and in particular the results shown in Example 5 showing the use of metalloproteinase inhibitors to treat wounds, the inventors expect that the invention used in this particular model would reduce the loss of mucosal crypts in the proximal small bowel and distal small bowel as detected by the area of the intact crypts per unit area of total mucosa compared to control groups. The inventors also expect the invention used in this particular model would reduce reduction in villi length compared to control groups. In summary, the inventors expected with a reasonable expectation of success, that the invention would prevent or accelerate repair of chemotherapy damage in the small bowel.

### EXAMPLE 9

Prophetic Example Demonstrating Methodologies to Study the Effectiveness of a Composition Enriched with a Property of Inhibiting Metalloproteinases as a Therapeutic Treatment for Gastric Ulcers.

The invention may be used to prevent or treat damage relating to gastric ulcers induced by non-steroidal anti-inflammatory drug (NSAID) therapy. The person skilled in the art will readily be able to investigate the claimed invention to prevent or treat gastric ulcers induced by NSAID therapy.

For example, adult Sprague Dawley rats gavaged with a high dose of a NSAID, indomethacin will be used as an experimental model of NSAIDs—induced gastritis. Control rats will receive no composition enriched with the property of inhibiting metalloproteinase whereas experimental rats will be either administered prophylatically with a composition enriched with the property of inhibiting metalloproteinases for 72, 48, 24 hours or 30 minutes prior to indomethacin treatment. Treated rats will be fed an unmodified diet and given a composition enriched with the property of inhibiting metalloproteinases by stomach gavage twice daily. Control rats will be fed an unmodified diet and gavaged with an equivalent volume of physiological saline. Treated and control rats will be fasted overnight and gavaged with 1 ml Indomethacin (100 mg/kg) to induce gastric ulceration. Both treated and control rats will be killed 5 hours post indomethacin gavage, for assessment of gastric ulceration.

The number, size and area per unit mucosal surface area of gastric mucosa damage, and cellular proliferation or apoptosis of cells in the gastric epithelium, induced by indomethacin treatment will be measured using techniques familiar to those skilled in the art and compared between control and treatment groups to determine the reduction or prevention or acceleration of repair, of gastric mucosa damage due to the use of compositions enriched with the property of inhibiting metalloproteinases. On the basis of the results shown in Examples 1A, 1B, 2, 3, and 5, the inventors expect with a reasonable expectation of success that the invention would reduce or prevent or accelerate the repair of damage to the gastric mucosa.

Finally, it is to be understood that various alterations, modifications and/or additions may be made without departing from the spirit of the present invention as outlined herein.

1. A composition derived directly or indirectly from a lactational secretion of an ungulate animal, the composition comprising an inhibitor of a metalloproteinase, wherein the inhibitor of metalloproteinase is enriched as compared with the lactational secretion from which the composition is derived.

2. A composition according to claim 1 wherein the metalloproteinase is selected from the group consisting of (i) the collagenases (metalloproteinases-1, 8 and 13); (ii) the gelatinases A and B (metalloproteinase-2 and metalloproteinase-9); (iii) the stromelysins 1 and 2 (metalloproteinases-3 and 10); (iv) matrilysin (MMP-7); enamelysin (MMP-20), macrophage metalloelastase (MMP12), and MMP-19 and (v) the membrane-type metalloproteinases (MT-MMP-1 to 4 and stromelysin-3, MMP-11).

3. A composition according to claim 1 wherein the inhibitor is a tissue inhibitor of a metalloproteinase (TIMP).

4. A composition according to claim 3 wherein the TIMP is a TIMP-2 polypeptide or a functional equivalent or fragment thereof.

5. A composition according to claim 4 wherein the TIMP-2 has a molecular weight of about 21,000 Da as determined by SDS-PAGE and/or has an isoelectric point of about 7.0.

6. A composition according to claim 5 wherein the TIMP-2 comprises the following N-terminal sequence: NH2-CSCSPVHP.

7. A composition according to claim 6 wherein the TIMP-2 comprises the following sequence:
NH2_CSCSPVHPQQAFCNADIVIRAKAVNKKEVDSGNDIYGNPIKRIQYEIKQIK MFKGPDQDIEFIYTAPAAAVCGVSLDIGGKKEYLIAGKAEGNGNMHITLCDFIVP WDTLSATQKKSLNHRYQMGCECKITRCPMIPCYISSPDECLWMDWVTEKNIN GHQAKFFACIKRSDGSCAWYRGAAPPKQEFLDIEDP_COOH,
or a functional equivalent or fragment thereof.

8. A composition according to claim 1 wherein the lactational secretion is milk or colostrum.

9. A composition according to claim 1 wherein the ungulate animal is selected from the group consisting of a cow, a sheep, a goat, a camel and a horse.

10. A composition according to claim 1 wherein if the composition is derived indirectly from a lactational secretion, an intermediate product is selected from the group consisting of cheese whey, skim milk, acid (casein) whey, colostral whey, defatted colostrum and dried milk powder or a combination thereof.

11. A composition according to claim 1 comprising a pharmaceutically, veterinarily, nutriceutically or cosmetically acceptable carrier and/or excipient.

12. A composition according to claim 1 wherein the inhibitor of a metalloproteinase is present at a purity of from about 70% to about 99% with respect to the total protein content of the composition.

13. A method for treating, preventing or ameliorating a disorder associated with undesirable metalloproteinase activity, the method including administering to an animal in need thereof an effective amount of a composition according to claim 1.

14. A method according to claim 13 wherein the disorder is a wound caused by pressure, vascular disease, diabetes, autoimmune disease, sickle cell diseases or hemophilia; a result of surgery; therapeutically induced; associated with disorders of the central nervous system, and resulting from any exfoliative disease of the skin; associated with either local or systemic infection such as yaws, HIV, chicken pox or herpes infection; congenital; a corneal injury to the eye; a pathological wound; a traumatic or accidental wound; or a burn.

15. A method according to claim 13 wherein the disorder is a dental or oral wound; peptic ulceration of the duodenum, stomach or esophagus; inflammatory bowel disease; an ulcer associated with stress conditions; damage to the lining of the alimentary tract; inadequate gut function or damage to the gut associated with prematurity; a diarrheal condition; a food intolerance; a cancer of the gastrointestinal tract; surgically induced damage to the gut; damage due to esophageal reflux; a condition associated with loss of gut barrier function; a congenital condition resulting in inadequate gastrointestinal function or damage; or an autoimmune disease that affects the gut.

16. A method according to claim 13 wherein the composition is administered so that the concentration of the inhibitor is present in a concentration of from about 0.1 ng/ml to about 10 μg/ml at the site of undesirable metalloproteinase activity.

17. A method according to claim 13 wherein the disorder is a disorder of the cardiovascular system selected from the group including dilated cardiomyopathy, congestive heart failure, atherosclerosis, plaque rupture, reperfusion injury, ischemia, chronic obstructive pulmonary disease, angioplastly restenosis, aortic aneurism; a disorder of a tissue where a metalloproteinase is involved in the irregular remodeling including disorders of bone, liver, lung and nervous tissues; a disorder relating to viral infection whereby metalloproteinase activity is altered; a disorder relating to inflammation involving the implication of metalloproteinases; a disorder relating to skin involving the implication of a metalloproteinase, including but not limited to psoriasis, scleroderma and atopic dermatitis or disorders relating to ultraviolet damage of skin which results in the skin having an aged and/or wrinkled appearance.

18. A method for at least partially purifying or enriching a metalloproteinase inhibitor, the method including the steps of;
providing a lactational secretion of an ungulate animal or a derivative of the lactational secretion, and
subjecting the lactational secretion or derivative to one or more treatment steps selected from the group consisting of centrifugation, micro-filtration, ultra-filtration, ion-exchange chromatography, molecular sieve chromatography, affinity chromatography, reverse-phase high performance liquid chromatography and transient acidification.

19. A method according to claim 18, the method comprising the steps of cation exchange chromatography followed by ultrafiltration.

20. A method according to claim 19 wherein the ultrafiltration step is followed by an acidification step.

21. A method according to claim 20 wherein the transient acidification step is followed by a gel filtration chromatography step.

22. A method according to claim 21 wherein the gel filtration step is followed by an anion exchange chromatography step.

23. A method according to claim 22 wherein the anion exchange chromatography step is followed by an affinity chromatography step.

24. A method according to claim 23 wherein the affinity chromatography step is followed by a reverse phase high performance liquid chromatography step.

25. A method according to claim 18, the method comprising a centrifugation step followed by an affinity chromatography step.
